(19) United States
(12) Patent Application Publication
(10) Pub. No.: US 2005/0260630 A1
(43) Pub. Date: Nov. 24, 2005

(54) Rapid methods for detecting methylation of a nucleic acid molecule

(75) Inventors: Jay I. Goodman, E. Lansing, MI (US); Ammie Norene Bachman, Stockbridge, MI (US)

Correspondence Address: WILMER CUTLER PICKERING HALE AND DORR LLP, 60 STATE STREET, BOSTON, MA, 02109, US

(73) Assignee: Michigan State University

(21) Appl. No.: 11/079,601

(22) Filed: Mar. 14, 2005

Related U.S. Application Data
(60) Provisional application No. 60/552,823, filed on Mar. 12, 2004.

(51) Int. Cl. C12Q 1/68; C12P 19/34

(52) U.S. Cl. 435/6; 435/91.2

(57) ABSTRACT

Disclosed are methods for determining the methylation status of a target double-stranded nucleic acid molecule using PCR amplification and capillary electrophoresis. The methods are generally useful in measuring the methylation status of a nucleic acid sample, including a mammalian genomic DNA sample, and may be further specifically applied to detecting changes in methylation status of a nucleic acid that are associated with exposure to a toxic compound or treatment, or that are associate with a disease or disorder.

### CROSS REFERENCE TO RELATED APPLICATIONS

This Application claims the benefit of priority to U.S. Provisional Application No. 60/552,823, filed Mar. 12, 2004.

### 1. FIELD OF THE INVENTION

The invention relates to biology and molecular biology. More specifically, the invention relates to methods for analyzing and identifying nucleic acid molecules.

### 2. BACKGROUND OF THE INVENTION

Methylation of cytosine residues of DNA is an epigenetic mechanism that regulates gene expression as well as tissue-specific, developmental, immunological and neurological processes (Robertson and Jones, Carcinogenesis 21(3): 461-467, 2000). Both hypo- and hypermethylation may lead to deleterious effects. In general, increases in methylation at promoter regions leads to transcriptional silencing by directly hindering the binding of transcription factors or by recruiting proteins that bind methylated cytosines, e.g., chromatin deacetylase (Attwood et al., Cell Mol. Life Sci. 59(2): 241-257, 2002). Conversely, hypomethylation may lead to the increased expression of certain genes and/or the loss of genomic stability via expression of transposable elements that are normally silenced by methylation (Counts and Goodman, Cell 83: 13-15, 1995; Carnell and Goodman, Toxicol. Sci. 75: 229-235, 2003).

Currently, detection of methylation of DNA is both time-consuming and costly. For example, methods for detecting methylation of DNA by reacting the DNA with bisulfite prior to sequencing have been described (Frommer. et al., Proc. Natl. Acad. Sci USA 89: 1827-1831, 1992; and Clark et al., Nucleic Acids Res. 22: 2990-2997, 1994). However, these methods are time-consuming and require detailed knowledge of the sequence being studied.

More recently, Gonzalgo et al. (U.S. Pat. No. 6,251,594) describe determining DNA methylation by reacting the DNA with sodium bisulfite to convert unmethylated residues to uracil, amplifying the DNA, and resolving the amplified DNA by polyacrylamide gel electrophoresis. A similar method involves digesting methylated DNA with a methylation-sensitive restriction endonuclease and resolving the digested, amplified DNA using polyacrylamide gel electrophoresis (Gonzalgo et al., Cancer Research 57: 594-599, 1997). FIG. 1 shows a representation of results from different control DNAs produced by this method of resolving amplified DNA by polyacrylamide gel electrophoresis. A comparison of treated DNA to a control DNA, such as that shown in this figure, enables a determination of whether the treated DNA is methylated, as indicated by the presence or absence of bands when DNA is digested with different restriction endonucleases prior to amplification. This figure was adapted from Watson and Goodman, Tox. Sci. 75: 289-299, 2003. However, because such methods involving polyacrylamide gel electrophoresis can only analyze a few samples at a time and resolution is limited, the number of PCR products resolved/identified is limited. Thus, these methods are both costly, time-consuming and yield a rather limited amount of data.

Given the crucial role that methylation of cytosine residues of DNA plays in the regulation of gene expression, there is a need for a DNA methylation detection method that is rapid, inexpensive, capable of detecting alterations in multiple regions of DNA simultaneously, and accurate.

### 3. SUMMARY OF THE INVENTION

The invention provides a DNA methylation detection method that is rapid, inexpensive, capable of detecting alterations (increases and/or decreases) in methylation in multiple regions of DNA simultaneously, and provides accurate, easily reproducible results.

Accordingly, in a first aspect, the invention provides methods for determining the methylation status of a target double-stranded nucleic acid molecule. The method includes contacting a target double-stranded nucleic acid molecule with a methylation-sensitive restriction endonuclease under conditions wherein the target double-stranded nucleic acid molecule is cleaved at a site recognized by the methylation-sensitive restriction endonuclease if the site is not methylated. Next, the target double-stranded nucleic acid molecule is PCR amplified with a detectably labeled primer that hybridizes to a predetermined region of the double-stranded nucleic acid molecule. The presence of the PCR product is next detected using capillary electrophoresis, where the presence of a product indicates that the target double-stranded nucleic acid molecule is methylated at the site recognized by the methylation-sensitive restriction endonuclease. In some embodiments, the detectably labeled primer is labeled with a fluorophore.

In some embodiments, the PCR amplification step includes amplification with a second primer that hybridizes to a second predetermined region of a second strand of the double-stranded nucleic acid molecule.

In some embodiments, the predetermined region is a GC rich region. In some embodiments, the predetermined region is a region on one of the strands of the double-stranded nucleic acid molecule. For example, the predetermined region may be within the 5′-flanking region (promoter region) of gene(s). In another example, the predetermined region may be at the 3′ end of one of the strands of the double-stranded nucleic acid molecule. In some embodiments, only a portion of the primer hybridizes to the predetermined region. For example, the 3′ end of a primer may be complementary (i.e., able to hybridize to) to the predetermined region of the double stranded nucleic acid molecule.

In some embodiments, the target double-stranded nucleic acid molecule is isolated from a prokaryotic cell or a eukaryotic cell including, without limitation, a mammalian cell an insect cell, or a plant cell. In some embodiments the target double-stranded nucleic acid molecule is genomic DNA. In some embodiments the target double-stranded nucleic acid molecule is mitochondrial DNA. In some embodiments, the target double-stranded nucleic acid molecule is heterologous to the cell.

In a further aspect, the invention provides another method for determining the methylation status of a double-stranded nucleic acid molecule. The method includes contacting the double-stranded nucleic acid molecule with a methylation-sensitive restriction endonuclease under conditions where the double-stranded nucleic acid molecules is cleaved at a site recognized by the methylation-sensitive restriction endonuclease if the site is not methylated. Next, the double-stranded nucleic acid molecule is PCR amplified with detectably labeled primer that hybridizes to a predetermined region of the nucleic acid molecule. Capillary electrophoresis is then used to detect the presence of a PCR product.

The method of this aspect also includes contacting the double-stranded nucleic acid molecule with a methylation-insensitive restriction endonuclease under conditions wherein the double-stranded nucleic acid molecule is cleaved at the same site recognized by the methylation-sensitive restriction endonuclease. In some embodiments, the method further includes contacting the double-stranded nucleic acid molecule with a methylation-insensitive restriction endonuclease under conditions wherein the double-stranded nucleic acid molecule is cleaved at a site other than the site recognized by the methylation-sensitive restriction endonuclease. The double-stranded nucleic acid molecule is next PCR amplified with the detectably labeled primer, and capillary electrophoresis is used to detect the presence of the PCR product.

The results of the two capillary electrophoresis analyses are then compared by comparing the methylation-sensitive digestion versus the methylation-insensitive digest (or by comparing the methylation-sensitive and insensitive double-digestion versus the methylation-insensitive digest), where a difference indicates that the double-stranded nucleic acid molecule is methylated at the site recognized by the methylation-sensitive restriction endonuclease. In some embodiments, the detectably labeled primer is labeled with a fluorophore.

In certain embodiments, the difference is an increase in the number of PCR products from the methylation-sensitive restriction endonuclease contacted nucleic acid molecule, as compared to the number of PCR products from the methylation-insensitive restriction endonuclease contacted nucleic acid molecule. In certain embodiments, the difference is a decrease in the number of PCR products from the methylation-sensitive restriction endonuclease contacted nucleic acid molecule, as compared to the number of PCR products from the methylation-insensitive restriction endonuclease contacted nucleic acid molecule.

In particular embodiments, the predetermined region to which the primer hybridizes is a GC rich region.

In some embodiments, the nucleic acid molecule is genomic DNA isolated from a cell, such as a mammalian cell. In particular embodiments, the cell has been contacted with a compound.

In another aspect, the invention provides a method for determining if a compound affects the methylation status of a cell. In this method a double-stranded nucleic acid molecule is isolated from a cell contacted with the compound, and the double-stranded nucleic acid molecule is contacted with a methylation-sensitive restriction endonuclease under conditions wherein the double-stranded nucleic acid molecule is cleaved at a site recognized by the methylation-sensitive restriction endonuclease if the site is not methylated. Next, the double-stranded nucleic acid molecule is PCR amplified with a detectably labeled primer that hybridizes to a predetermined region of a strand of the double-stranded nucleic acid molecule, and the PCR product detected using capillary electrophoresis. A double-stranded nucleic acid molecule isolated from a cell not contacted with the compound is also digested with the methylation-sensitive restriction endonuclease and PCR amplified. The PCR product is detected using capillary electrophoresis. The PCR products from the two cells (i.e., the cell contacted with the compound and the cell not contacted with the compound) are compared, a difference indicating that the compound affects the methylation status of the cell. In some embodiments, the detectably labeled primer is labeled with a fluorophore.

In certain embodiments, the nucleic acid molecule is genomic DNA. In particular embodiments, the predetermined region to which the primer hybridizes is a GC-rich region.

In some embodiments, the difference is an increase in the number of PCR products from the compound-contacted cell as compared to the cell not contacted with the compound. In other embodiments, the difference is a decrease in the number of PCR products from the compound-contacted cell as compared to the cell not contacted with the compound.

In some embodiments, the compound enhances the proliferation of the cell. In certain embodiments, the compound is a carcinogen. In certain embodiments, the compound abrogates the growth of the cell. In particular embodiments, the compound is toxic to the cell.

In a further aspect, the invention provides a method for determining an indication of the level of expression of a target nucleic acid molecule by a cell. The method of this aspect includes contacting the target double-stranded nucleic acid molecule isolated from the cell with a methylation-sensitive restriction endonuclease under conditions wherein the target double-stranded nucleic acid molecule is cleaved at a site recognized by the methylation-sensitive restriction endonuclease if the site is not methylated. Next, the target double-stranded nucleic acid molecule is PCR amplified with a detectably labeled primer that that hybridizes to a predetermined region of a strand of the double-stranded nucleic acid molecule, and the PCR product detected by capillary electrophoresis. The number of PCR products may be viewed as being inversely related to the level of expression of the target double-stranded nucleic acid molecule by the cell. In some embodiments, the detectably labeled primer is labeled with a fluorophore.

In certain embodiments, the predetermined region is a region on one of the strands of the double-stranded nucleic acid molecule. For example, the predetermined region may be at the 3′ end of one of the strands of the double-stranded nucleic acid molecule. In some embodiments, the PCR amplification step includes amplification with a second primer that hybridizes to a second predetermined region of a second strand of the double-stranded nucleic acid molecule.

In certain embodiments, the target double-stranded nucleic acid molecule is isolated from a cell, such as a mammalian cell. In particular embodiments the target double-stranded nucleic acid molecule is genomic DNA. In some embodiments, the target double-stranded nucleic acid molecule is heterologous to the cell.

### 4. DESCRIPTION OF THE DRAWINGS

FIG. 1 is a photographic representation of a polyacrylamide gel showing the prior art method of resolution of methylation-sensitive restriction endonuclease digested, PCR amplified DNA using polyacrylamide gel electrophoresis. The four lanes show different control mouse liver DNAs which were digested, PCR amplified, and then resolved by polyacrylamide gel electrophoresis.

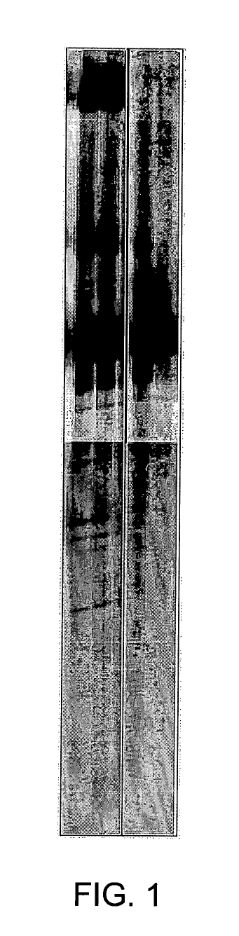

FIG. 2 is a graphic representation of data using from the analysis of murine liver genomic DNA digested, prior to PCR amplification, with RsaI and MspI using capillary electrophoresis. The individual data points are expressed as base pairs vs. peak area. The larger the peak area, the more fragments of that base pair size (in length) are present.

FIG. 3 is a graphic representation of data from the analysis of murine liver genomic DNA digested, prior to PCR amplification, with RsaI and HpaII using capillary electrophoresis. The individual data points are expressed as base pairs vs. peak area. The larger the peak area, the more fragments of that base pair size (in length) are present.

FIG. 4 is a graphic representation showing the average percentage of PCR products formed when DNA was digested with RsaI and HpaII prior to PCR as compared with the average percentage of PCR products formed when DNA was digested with RsaI and MspI prior to PCR. The data are presented as average MspI—Average HpaII)/Average HpaII)×100. The positive values shown on the graph indicate less cutting by MspI than HpaII, while negative values represented more cutting by MspI than HpaII.

FIG. 5 is a graphic representation showing RsaI/HpaII digest, following arbitrarily primed PCR (AP-PCR) as capillary electrophoresis (CE) data output in terms of the consensus treated as a percent of the consensus control. The asterisks denote a significant difference between the control mean and treated mean for that particular size PCR product found by conducting a t-test where α=0.05.

FIG. 6 is a graphic representation showing the effects of high dose (27 mg CSC) promotion on the methylation of GC rich regions. RsaI/MspI digest, arbitrarily primed PCR and capillary electrophoresis was performed on DNA isolated from SENCAR control (Acetone) or treated (27 mg CSC) mice. The asterisks denote a significant difference between the control mean and treated mean for that size PCR product where p<0.05 in the Student's t-test.

FIG. 7 is a graphic representation showing sites of new methylation following high dose promotion (27 mg CSC). RsaI/MspI digest, arbitrarily primed PCR and capillary electrophoresis was performed on DNA isolated from SENCAR control (Acetone) or treated (27 mg CSC) mice. Promotion with 27 mg CSC for 8 wks yielded 27 sites of new methylation.

FIG. 8 is a graphic representation showing the effects of high dose (27 mg CSC) promotion on GC rich region methylation. RsaI/HpaII digest, arbitrarily primed PCR and capillary electrophoresis was performed on DNA isolated from SENCAR control (Acetone) or treated (27 mg CSC) mice. Promotion with 27 mg CSC for 8 wks yielded 2 sites of hypomethylation and 1 site of new methylation.

FIG. 9 is a graphic representation showing the site of new methylation following high dose promotion (27 mg CSC). RsaI/HpaII digest, arbitrarily primed PCR and capillary electrophoresis was performed on DNA isolated from SENCAR control (Acetone) or treated (27 mg CSC) mice. Promotion with 27 mg CSC for 8 wks yielded 1 site of new methylation.

FIG. 10 is a graphic representation showing the effect of hypertension on the methylation status of GC-rich regions of DNA. RsaI/HpaII digest, arbitrarily primed PCR and capillary electrophoresis was performed on DNA isolated from the aortas of control and hypertensive rats. The data are expressed in terms of the hypertensive mean (consensus hypertensive) for each PCR product size as a percent of the control mean (consensus control) for each PCR product size. Positive values indicate sites of hypermethylation while negative values indicate sites of hypomethylation. Only those values that are significantly different from control are considered to be “changes,” Student's t-test, p<0.05.

FIG. 11 is a graphic representation showing the effect of hypertension on the methylation status of GC-rich regions of DNA. Sites of new methylation were investigated using an RsaI/HpaII digest and subsequent AP-PCR, followed by separation of the products by capillary electrophoresis, on DNA isolated from the aortas of control and hypertensive rats. The data presented indicate sites of new methylation, i.e., sites that were methylated in the treated animals but not in the controls.

FIG. 12 is a graphic representation showing the effect of hypertension on the methylation status of GC-rich regions of DNA. Sites of hypomethylation and hypermethylation associated with hypertension were investigated using a RsaI/MspI digest. An RsaI/MspI digest and subsequent AP-PCR, followed by separation of the products by capillary electrophoresis, was performed on DNA isolated from the aortas of control and hypertensive rats. The data is expressed in terms of the hypertensive mean (consensus hypertensive) for each PCR product size as a percent of the control mean (consensus control) for each PCR product size. Positive values indicate sites of hypermethylation while negative values indicate sites of hypomethylation. Only those values that are significantly different from control are considered to be “changes,” Student's t-test, p<0.05.

FIG. 13 is a graphic representation showing the effect of hypertension on the methylation status of GC-rich regions of DNA. Sites of new methylation were investigated using an RsaI/MspII digest. An RsaI/MspI digest and subsequent AP-PCR, followed by separation of the products by capillary electrophoresis, was performed on DNA isolated from the aortas of control and hypertensive rats. The data presented indicate sites of new methylation, i.e., sites that were methylated in the treated animals but not in the controls.

### 5. DETAILED DESCRIPTION OF THE INVENTION

The patent and scientific literature referred to herein establishes knowledge that is available to those with skill in the art. The issued U.S. patents, allowed applications, published foreign applications, and references, including GenBank database sequences, that are cited herein are hereby incorporated by reference to the same extent as if each was specifically and individually indicated to be incorporated by reference.

The present invention stems from the inventors' discovery that methylation analysis of nucleic acid molecules can be performed using capillary electrophoresis. The results of this new method are surprising accurate, rapid, and cost-effective.

Aspects of the invention provide methods for rapidly identifying the methylation status in nucleic acid molecules, including the simultaneous assessment of the methylation status in multiple regions of DNA. These methods are useful for quickly detecting methylation in a given nucleic acid molecule, or for detecting changes in methylation patterns in a large number of nucleic acid molecules (e.g., in genomic DNA). The disclosed methods are useful, e.g., for identifying a compound, such as a chemical or a group of chemicals, that affects the pattern of methylation, a process which may affect the amount of expression of a given nucleic acid molecule. Where the methods of the invention are used to detect methylation patterns in a large number of nucleic acid molecules, such as the genomic DNA of a cell, a change may indicate a change in the development or health of that cell.

Accordingly, in a first aspect, the invention provides methods for determining the methylation status of a target double-stranded nucleic acid molecule. As used herein, “nucleic acid” or “nucleic acid molecule” means any deoxyribonucleic acid (DNA) or ribonucleic acid (RNA), including, without limitation, complementary DNA (cDNA), genomic DNA, RNA, hnRNA, messenger RNA (mRNA), DNA/RNA hybrids, or synthetic nucleic acids (e.g., an oligonucleotide) comprising ribonucleic and/or deoxyribonucleic acids or synthetic variants thereof (e.g., nucleic acids having other than phosphodiester internucleoside linkages). The nucleic acid molecule of the invention may be from any source including, without limitation, the nucleus of a eukaryotic cell (e.g., genomic DNA), mitochondria (e.g., mitochondrial DNA), and a prokaryotic cell. The nucleic acid molecule of the invention includes, without limitation, an oligonucleotide or a polynucleotide. The nucleic acid molecule can be single-stranded or partially or completely double-stranded (duplex). Duplex nucleic acid molecule s can be homoduplex or heteroduplex.

The method, in accordance with one aspect of the invention, includes contacting a target double-stranded nucleic acid molecule with a methylation-sensitive restriction endonuclease under conditions wherein the target double-stranded nucleic acid molecule is cleaved at a site recognized by the methylation-sensitive restriction endonuclease if the site is not methylated (e.g., at a particular nucleotide, such as cytosine, that affects the ability of the enzyme to cleave the nucleic acid molecule). Next, the target double-stranded nucleic acid molecule is PCR amplified with a detectably labeled primer that hybridizes to a predetermined region of a strand of the double-stranded nucleic acid molecule. Thus, a PCR product will be formed only if the target double-stranded nucleic acid molecule was not cleaved by the methylation-sensitive restriction endonuclease. In other words, no PCR product will be formed if the double-stranded nucleic acid molecule is not methylated at the site recognized by the methylation-sensitive restriction endonuclease, because the target nucleic acid molecule will be cleaved at the site, thereby destroying the template for the PCR reaction. The presence of the PCR product is next detected using capillary electrophoresis, where the presence of a product indicates that the target double-stranded nucleic acid molecule is methylated at the site recognized by the methylation-sensitive restriction endonuclease.

In a further aspect, the invention provides a method for determining the methylation status of a double-stranded nucleic acid molecule. The method includes contacting the double-stranded nucleic acid molecule with a methylation-sensitive restriction endonuclease under conditions where the double-stranded nucleic acid molecules is cleaved at a site recognized by the methylation-sensitive restriction endonuclease if the site is not methylated. Next, the double-stranded nucleic acid molecule is PCR amplified with detectably labeled primer that hybridizes to a predetermined region of the nucleic acid molecule. Capillary electrophoresis is then used to detect the presence of a PCR product.

The method of this aspect also includes contacting the double-stranded nucleic acid molecule with a methylation-insensitive restriction endonuclease under conditions wherein the double-stranded nucleic acid molecule is cleaved at the same site recognized by the methylation-sensitive restriction endonuclease. The double-stranded nucleic acid molecule is next PCR amplified with the detectably labeled primer, and then capillary electrophoresis is used to detect the presence of the PCR product.

The results of the two capillary electrophoresis analyses are next compared (i.e., comparing the results of the methylation-sensitive digestion versus the results of the methylation-insensitive digest), where a difference indicates that the double-stranded nucleic acid molecule is methylated at the site recognized by the methylation-sensitive restriction endonuclease.

In an alternative, the method further comprises digesting the double-stranded nucleic acid molecule with a methylation-insensitive restriction endonuclease that cleaves the nucleic acid molecule at a site other than the site recognized by the methylation-sensitive restriction endonuclease prior to PCR amplification. The results of the two capillary electrophoresis analyses are next compared (i.e., comparing the results of the methylation-sensitive and methylation-insensitive double-digestion versus the results of the methylation-insensitive digest), where a difference indicates that the double-stranded nucleic acid molecule is methylated at the site recognized by the methylation-sensitive restriction endonuclease.

Capillary electrophoresis refers to an automated analytical technique that separates particles by applying voltage across buffer filled capillaries. The capillaries are typically fused silica capillaries with an inner diameter of about 50-100 μm, and about 30-80 cm in length. The capillaries are filled with a sieving matrix of a gel material and electrophoresis buffer. The migration speed of particles through capillaries is based on the particle size and charge under the influence of applied voltage. The particles are seen as peaks as they pass through the detector and the area of each peak is proportional to the concentration of the particle, which allows quantitative determinations. Some advantages of capillary electrophoresis include low cost (once the initial investment in a capillary electrophoresis device is made), less labor-intensive, fine resolution of the particles, high separation efficiency (10⁵ to 10⁶ theoretical plates), small sample size required (1-10 μl), fast separation (1 to 45 minutes, depending upon the complexity of the separation), easy and predictable selectivity, automation, provides data in a manner that is easily quantifiable, good reproducibility, and the ability to be readily coupled with a mass spectrophotometer.

In some nonlimiting embodiments, a capillary electrophoresis instrument may contain fiber optical detection systems, high capacity autosamplers, and temperature control devices. In one embodiment, detection is by ultraviolet (UV) absorbance (e.g., with a diode array). Thus, in particular embodiments, the particles are detectably labeled, and so are more easily detected by a capillary electrohoresis. In some embodiments, the particles (e.g., nucleic acid molecules or PCR products) are detectably labeled with a fluorophore. Other commercial detectors include fluorescence detection and coupling to mass spectrometers. Indirect UV detection is widely used for detecting solutes having no chromophores such as metal ions or inorganic anions. Low UV wavelengths (e.g., 190-200 nm) are also used to detect simple compounds such as organic acids.

The data from a capillary electrophoresis device are collected and stored by computer, and analyzed using numerous commercially available computer programs. Moreover, capillary electrophoresis instruments with numerous capillaries (e.g., up to 384 capillaries) are commercially available (e.g., from Applied Biosystems, Foster City, Calif.). In addition, microchip capillary electrophoresis devices may also be used. Thus, capillary electrophoresis allows high throughput screening of numerous samples quickly, and cost-effectively.

As used herein, “capillary electrophoresis” also includes the technique of capillary electrochromatography (CEC), which is a hybrid of capillary electrophoresis and a hybrid of CE and high performance liquid chromatography (HPLC) and achieves chromatographic separations using capillaries packed with stationary phase. The solvent is pumped through the electro-osmotic flow (EOF) when the voltage is applied. Particles interact differentially with the stationary phase and are separated in a manner similar to HPLC. Because the EOF does not generate back-pressure, a small stationary phase (1-3 mm) can be used and this increases peak efficiency. In addition, separation efficiency increases because the flow profile of the EOF is flat and there is less dispersion than with a pump. This improved separation efficiency gives sharper peaks that give better resolution, or faster separations, compared to conventional HPLC separations.

Capillary electrophoresis has been used widely to separate DNA (see, e.g., Slater et al., Curr. Opin. Biotechnol. 14: 58-64, 2003; Altria and Elder, J. Chromatog. A 1023: 1-14, 2004).

By “methylation-sensitive restriction endonuclease” is meant a restriction endonuclease (also called a restriction enzyme) that does not cleave a nucleic acid molecule substrate if one or more of the bases in the recognition site of the restriction endonuclease is methylated. For example, the HpaII restriction endonuclease will not cleave its recognition site, 5′CCGG 3′ if the internal C (i.e., the C adjacent to the 5′ G) is methylated. By contrast, a “methylation-insensitive restriction endonuclease” will cleave a nucleic acid molecule substrate at the restriction endonuclease's recognition site regardless of whether or not one or more bases in its recognition site is methylated. Non-limiting examples of methylation-sensitive restriction endonucleases include MboI, EagI, NruI, HpaII, MspI and HhaI. Of course, a restriction endonuclease (either methylation-sensitive or methylation-insensitive) will cleave a target nucleic acid molecule bearing its recognition site under conditions (e.g., in appropriate salt concentration, at appropriate temperature) where the restriction endonuclease is enzymatically active. Restriction endonucleases are typically commercially available (e.g., from New England Biolabs, Beverly, Mass.), and come supplied with a reaction buffer in which the restriction endonuclease is enzymatically active, and directions for using the restriction endonuclease (e.g., appropriate digestion temperature, length of time for digestion). Other sources of information for conditions under which a restriction endonuclease is enzymatically active are known (see, e.g., Ausubel et al., supra).

As used herein, “hybridize” means the base-specific hydrogen bonding between complementary strands of nucleic acid molecules, preferably to form Watson-Crick or Hoogsteen base pairs, although other modes of hydrogen bonding, as well as base stacking can lead to hybridization. Accordingly, a primer hybridizes to a nucleic acid molecule if it is able to form base-specific hydrogen bonding with the nucleic acid molecule (or if it is able to form base-specific hydrogen bonding with one strand of a double-stranded nucleic acid molecule). In certain embodiments, the primer is incompletely complementary to the target nucleic acid molecule (i.e., not every base in the primer forms a hydrogen bond with a corresponding base in the nucleic acid molecule). In some embodiments, a primer that is incompletely complementary to the target nucleic acid molecule hybridizes to the nucleic acid molecule under stringent conditions. In accordance with the invention, stringent conditions are selected to be about 5° C. lower than the thermal melting point (Tₘ) for a specific sequence at a defined ionic strength and pH. The Tₘ is the temperature (under defined ionic strength and pH) at which 50% of the target sequence hybridizes to a perfectly matched sequence. Typical stringent conditions are those in which the salt concentration is at least about 0.02 M at pH 7 and the temperature is at least about 60° C.

By “PCR” or “polymerase chain reaction” is meant a method for amplifying a double-stranded nucleic acid molecule using a polymerase, sufficient bases (i.e., dATP, dCTP, dGTP, and dTTP), and at least one primer that is complementary to each strand of the nucleic acid molecule. Because the newly synthesized DNA strand can subsequently serve as additional templates for the same primer, successive rounds of primer annealing, strand elongation, and dissociation produce rapid and highly specific amplification of the desired nucleic acid molecule. The polymerase chain reaction is well known (see, e.g., Ausubel et al., Current Protocols in Molecular Biology, John Wiley & Sons, New York, N.Y., 1994-2004 (updated regularly) and Sambrook and Russell., Molecular Cloning. A Laboratory Manual, 3^{rd} Ed., Cold Spring Harbor Laboratory, Cold Spring Harbor, 2001).

As used herein, by “detectably labeled” is meant that a primer is attached (either covalently or noncovalently) to a chemical that can be detected, either by the human eye, or by machine. In certain embodiments, the label is an enzyme (which is detectable by virtue of its enzymatic activity). In certain embodiments, the label is a chromophore as a fluorophore. “Fluorophore” is used herein to mean a protein or chemical that glows at a particular, readable color of light when it is excited by ultraviolet light of a particular wavelength. A primer to which a fluorophore is attached is said to be “detectably labeled.” Non-limiting examples of useful fluorophores are listed in Table I, and are commercially available, for example, from Sythergen or Integrated DNA Technologies.

TABLE I
Representative Fluorophores

| | Formula | | | | |
|---|---|---|---|---|---|
| | Weight | Absorbance | Emission | Extinction | Fluorescent |
| Fluorescent Dye | (g/mol) | (nm) | (nm) | Coefficient | Color |
| Tamra-dT | 870.9 | 544 | 576 | | Yellow- |
| | | | | | Orange |
| 5-Fluorescein (FITC) | 537.6 | 495 | 520 | 73000 | Yellow- |
| | | | | | Green |
| 5-Carboxyfluorescein | 358.0 | 495 | 520 | 83000 | Yellow- |
| (FAM) | | | | | Green |
| 6-Carboxyfluorescein | 537.5 | 495 | 520 | 83000 | Yellow- |
| (FAM) | | | | | Green |
| 3′ 6-Carboxyfluorescein | 569.5 | 495 | 520 | 83000 | Yellow- |
| (FAM) | | | | | Green |
| 6-Carboxyfluorescein- | 537.5 | 495 | 520 | 83000 | Yellow- |
| DMT (FAM-X) | | | | | Green |
| 5(6)-Carboxyfluorescein | 537.5 | 495 | 520 | 83000 | Yellow- |
| (FAM) | | | | | Green |
| 6-Hexachlorofluorescein | 744.1 | 535 | 556 | 73000 | Yellow |
| (HEX) | | | | | |
| 6-Tetrachlorofluorescein | 675.2 | 521 | 536 | 73000 | Yellow- |
| (TET) | | | | | Green |
| JOE | 487.0 | 520 | 548 | 73000 | Yellow |
| LightCycler Red 640 | 758.0 | 625 | 640 | | Red |
| LightCycler Red 705 | 753.0 | 685 | 705 | | Red |
| FAR-Fuchsia (5′-Amidite) | 776.0 | 567 | 597 | 150000 | Yellow- |
| | | | | | Orange |
| FAR-Fuchsia (SE) | 776.0 | 567 | 597 | 150000 | Yellow- |
| | | | | | Orange |
| FAR-Blue (5′-Amidite) | 824.0 | 660 | 678 | 150000 | Red |
| FAR-Blue (SE) | 824.0 | 660 | 678 | 150000 | Red |
| FAR-Green One (SE) | 976.0 | 800 | 820 | 130000 | Near-IR |
| FAR-Green Two (SE) | 960.0 | 772 | 788 | 150000 | Near-IR |
| Oregon Green 488 | 394.0 | 496 | 516 | 76000 | Yellow- |
| | | | | | Green |
| Oregon Green 500 | 431.0 | 499 | 519 | 84000 | Yellow- |
| | | | | | Green |
| Oregon Green 514 | 494.0 | 506 | 526 | 85000 | Yellow- |
| | | | | | Green |
| BODIPY FL-X | 387.0 | 504 | 510 | 70000 | Green |
| BODIPY FL | 273.8 | 504 | 510 | 70000 | Green |
| BODIPY-TMR-X | 493.0 | 544 | 570 | 56000 | Yellow |
| BODIPY R6G | 322.0 | 528 | 547 | 70000 | Yellow |
| BODIPY 650/665 | 529.5 | 650 | 665 | 101000 | Red |
| BODIPY 564/570 | 348.0 | 563 | 569 | 142000 | Yellow |
| BODIPY 581/591 | 374.0 | 581 | 591 | 136000 | Yellow- |
| | | | | | Orange |
| BODIPY TR-X | 519.0 | 588 | 616 | 68000 | Red- |
| | | | | | Orange |
| BODIPY 630/650 | 545.5 | 625 | 640 | 101000 | Red |
| BODIPY 493/503 | 302.0 | 500 | 509 | 79000 | Green |
| Carboxyrhodamine 6G | 441.0 | 524 | 557 | 102000 | Yellow |
| MAX | 441.0 | 525 | 555 | 102000 | Yellow |
| 5(6)- | 412.5 | 546 | 576 | 90000 | Yellow- |
| Carboxytetramethylrhodamine | | | | | Orange |
| (TAMRA) | | | | | |
| 6- | 413.0 | 544 | 576 | 90000 | Yellow- |
| Carboxytetramethylrhodamine | | | | | Orange |
| (TAMRA) | | | | | |
| 5(6)-Carboxy-X- | 516.7 | 576 | 601 | 82000 | Orange |
| Rhodamine (ROX) | | | | | |
| 6-Carboxy-X-Rhodamine | 517.0 | 575 | 602 | 82000 | Orange |
| (ROX) | | | | | |
| AMCA-X (Coumarin) | 328.0 | 353 | 442 | 19000 | Blue |
| Texas Red-X | 702.0 | 583 | 603 | 116000 | Orange |
| Rhodamine Red-X | 654.0 | 560 | 580 | 129000 | Yellow- |
| | | | | | Orange |
| Marina Blue | 252.3 | 362 | 459 | 19000 | Blue |
| Pacific Blue | 224.2 | 416 | 451 | 37000 | Blue |
| Rhodamine Green-X | 394.0 | 503 | 528 | 74000 | Yellow- |
| | | | | | Green |
| 7-diethylaminocoumarin- | 243.0 | 432 | 472 | 56000 | Blue- |
| 3-carboxylic acid | | | | | Green |
| 7-methoxycoumarin-3- | 202.0 | 358 | 410 | 26000 | Violet |
| carboxylic acid | | | | | |
| Cy3 | 508.6 | 552 | 570 | 150000 | Yellow |
| Cy3B | 543.0 | 558 | 573 | 130000 | Yellow- |
| | | | | | Orange |
| Cy5 | 534.6 | 643 | 667 | 250000 | Red |
| Cy5.5 | 634.8 | 675 | 694 | 250000 | Red |
| DY-505 | 0.0 | | 505 | | Green |
| DY-550 | 667.8 | 553 | 578 | 122000 | Yellow |
| DY-555 | 636.2 | 555 | 580 | 100000 | Yellow- |
| | | | | | Orange |
| DY-610 | 667.8 | 606 | 636 | 140000 | Red |
| DY-630 | 634.8 | 630 | 655 | 120000 | Red |
| DY-633 | 751.9 | 630 | 659 | 120000 | Red |
| DY-636 | 760.9 | 645 | 671 | 120000 | Red |
| DY-650 | 686.9 | 653 | 674 | 77000 | Red |
| DY-675 | 706.9 | 674 | 699 | 110000 | Red |
| DY-676 | 808.0 | 674 | 699 | 84000 | Red |
| DY-681 | 736.9 | 691 | 708 | 125000 | Red |
| DY-700 | 668.9 | 702 | 723 | 96000 | Red |
| DY-701 | 770.9 | 706 | 731 | 115000 | Red |
| DY-730 | 660.9 | 734 | 750 | 113000 | Red |
| DY-750 | 713.0 | 747 | 776 | 45700 | Near-IR |
| DY-751 | 912.1 | 751 | 779 | 220000 | Near-IR |
| DY-782 | 660.9 | 782 | 800 | 102000 | Near-IR |
| Cy3.5 | 576.7 | 581 | 596 | 150000 | Yellow- |
| | | | | | Orange |
| EDANS | 307.1 | 336 | 490 | 5700 | Blue- |
| | | | | | Green |
| WellRED D2-PA | 611.0 | 750 | 770 | 170000 | Red |
| WellRED D3-PA | 645.0 | 685 | 706 | 224000 | Red |
| WellRED D4-PA | 544.8 | 650 | 670 | 203000 | Red |
| Pyrene | 535.6 | 341 | 377 | 43000 | Violet |
| Cascade Blue | 580.0 | 399 | 423 | 30000 | Violet |
| Cascade Yellow | 448.5 | 409 | 558 | 24000 | Yellow |
| PyMPO | 467.4 | 415 | 570 | 26000 | Yellow |
| Lucifer Yellow | 605.5 | 428 | 532 | 11000 | Yellow- |
| | | | | | Green |
| NBD-X | 276.3 | 466 | 535 | 22000 | Yellow- |
| | | | | | Green |
| Carboxynapthofluorescein | 458.5 | 598 | 668 | 42000 | Red |
| Alexa Fluor 350 | 295.4 | 346 | 442 | 19000 | Blue |
| Alexa Fluor 430 | 586.8 | 434 | 541 | 16000 | Yellow |
| Alexa Fluor 488 | 528.4 | 495 | 519 | 71000 | Yellow- |
| | | | | | Green |
| Alexa Fluor 532 | 608.8 | 532 | 554 | 81000 | Yellow |
| Alexa Fluor 546 | 964.4 | 556 | 573 | 104000 | Yellow- |
| | | | | | Orange |
| Alexa Fluor 555 | 850.0 | 555 | 565 | 150000 | Yellow |
| Alexa Fluor 568 | 676.8 | 578 | 603 | 91300 | Orange |
| Alexa Fluor 594 | 704.9 | 590 | 617 | 73000 | Red- |
| | | | | | Orange |
| Alexa Fluor 633 | 1085.0 | 632 | 647 | 100000 | Red |
| Alexa Fluor 647 | 850.0 | 650 | 665 | 239000 | Red |
| Alexa Fluor 660 | 985.0 | 663 | 690 | 132000 | Red |
| Alexa Fluor 680 | 1035.0 | 679 | 702 | 184000 | Red |
| Alexa Fluor 700 | 1285.0 | 702 | 723 | 192000 | Red |
| Alexa Fluor 750 | 1185.0 | 749 | 775 | 240000 | Near-IR |
| Oyster 556 | 850.0 | 556 | 570 | 155000 | Yellow |
| Oyster 645 | 1000.0 | 645 | 666 | 250000 | Red |
| Oyster 656 | 900.0 | 656 | 674 | 220000 | Red |
| 5(6)-Carboxyeosin | 689.0 | 521 | 544 | 95000 | Yellow |
| Erythrosin | 814.0 | 529 | 544 | 90000 | Yellow |

In some embodiments, the predetermined region of the strand of the double-stranded nucleic acid molecule is a GC rich region in the nucleic acid molecule. Thus, the detectably labeled primer can hybridize arbitrarily to either strand of the target double-stranded nucleic acid molecule. This embodiment is particularly useful where the sequence of the target double-stranded nucleic acid molecule is not known. Where the sequence of the double-stranded nucleic acid molecule is known, the detectably labeled primer can be designed to be complementary to one of the strands of the double-stranded nucleic acid molecule. In one non-limiting example, the detectably labeled primer can be designed to be complementary to a region within the 5′-flanking region (promoter region) of gene(s). The primer may also be complementary to the 3′ end of one of the strands of the double-stranded nucleic acid molecule.

In some embodiments, only a portion of the primer hybridizes to the predetermined region. For example, the 3′ end of a primer may be complementary (i.e., able to hybridize to) to the predetermined region of the double stranded nucleic acid molecule. In some embodiments, the double-stranded nucleic acid molecule contacted with the methylation-sensitive restriction endonuclease is PCR amplified with a first detectably labeled primer that hybridizes to a predetermined region of a strand of the double-stranded nucleic acid molecule and a second primer that hybridizes to a second predetermined region of a second strand of the double-stranded nucleic acid molecule. Note that because the first primer is detectably labeled, the second primer need not be labeled. In one variation of this embodiment, either the first or the second primer, or both, can hybridize to a GC rich region in the nucleic acid molecule. In particular variations the detectably labeled first primer hybridizes to one strand of the double-stranded nucleic acid molecule and the second primer hybridizes to the other strand of the double-stranded nucleic acid molecule.

In certain embodiments, the target double-stranded nucleic acid molecule is isolated from a cell, such as a bacterial cell or a mammalian cell. Non-limiting mammalian cells of the invention include cells from a primate (e.g., a human or a baboon), a laboratory animal (e.g., a mouse or rat), a livestock animal (e.g., a pig or a cow), or a domesticated animal (e.g., a dog or a cat).

As used herein, “isolated” refers to a nucleic acid molecule separated from other molecules (e.g., protein, carbohydrates, lipids, and other nucleic acid molecules) that are present in the natural source of the nucleic acid molecule. For example, a nucleic acid molecule isolated from a murine liver cell is separated from the other molecules present in that murine liver cell, such that the isolated nucleic acid molecule is substantially free of other molecules present in murine liver cells. Thus, the term “isolated” also refers to a nucleic acid molecule that is substantially free of cellular material, viral material, or culture medium when produced by recombinant DNA techniques, or chemical precursors or other chemicals when chemically synthesized. By “substantially free” is meant at least about, or at least about 75%, or at least about 85%, or at least about 90%, or at least about 95% pure, i.e., free from other organic molecules with which it naturally occurs and free from materials used during the purification process. Methods for isolating nucleic acid molecules from cells are well known (see, e.g., Ausubel et al., supra).

In some embodiments, the methods of the invention are useful for screening cells that have been introduced, either transiently or stably, with a nucleic acid molecule encoding a protein of interest, where a clone expressing a large amount of protein is desired. As used herein, “introduced” means that the nucleic acid molecule has been inserted into the cell by any means including, without limitation, transfection, transformation, and viral infection. Where a nucleic acid molecule is introduced into a cell, that nucleic acid molecule is “heterologous” to the cell, even if the origin of species of the nucleic acid molecule is the same as that of the cell (e.g., murine fibronectin-encoding nucleic acid molecule is heterologous to a murine cell if the murine fibronectin-encoding nucleic acid molecule was introduced into that cell). Thus, in some embodiments of the invention, the target double-stranded nucleic acid molecule is heterologous to the cell.

To produce recombinant proteins, it is routine to introduce cells (e.g., mammalian cells, such as CHO cells or HeLa cells) with a target nucleic acid molecule encoding the protein of interest. In some embodiments, the target nucleic acid molecule is positioned for expression in the cell, for example, by incorporating into the cell's genomic DNA in an appropriate location such that the target nucleic acid molecule is expressed by the cell (e.g., the target nucleic acid molecule incorporated 3′ of a promoter sequence in the cell's genome). In some embodiments, the target nucleic acid molecule is positioned for expression by its incorporation into an expression plasmid or vector. As used herein, by “expression plasmid” or “expression vector” refers to a vector or plasmid in which a nucleic acid molecule encoding a protein of interest is operably linked to regulatory sequences (e.g., promoters, enhancers), such that a cell introduced with the expression vector or expression plasmid expresses the protein of interest encoded by the target nucleic acid molecule. Such an expression plasmid may be circular, or may be linearized, prior to introduction into the cell. Non-limiting useful expression vectors include recombinant viruses, such as vaccinia virus, adenovirus, and lentivirus.

Once the nucleic acid molecule, or an expression plasmid or vector containing the nucleic acid molecule positioned for expression is introduced into the cell, it is routine to screen individual clones for their ability to express the protein. The methods of the invention are useful for quickly screening numerous clones to identify those that have low amounts of methylation of the introduced target nucleic acid molecule, and therefore express high amounts of the protein of interest.

In one non-limiting example, CHO cells (commercially available from the American Type Culture Collection, Manassas, Va.) are transfected (e.g., by electroporation) with a linearized expression plasmid containing a nucleic acid molecule encoding human insulin, and stable clones generated. The clones are then screened for an ability to secrete a high amount of insulin. However, with each passing generation, the amount of insulin secreted by the cell clones may diminish. This reduction in insulin expression may be due to the site in the genome in which the expression plasmid integrated. This reduction may also be due to methylation of the heterologous insulin-encoding nucleic acid molecule. Using the methods of the invention, those clones that have low levels of methylation of the heterologous insulin-encoding nucleic acid molecule are selected.

In some embodiments, the target nucleic acid molecule is genomic DNA isolated from a cell. For example, it may be desirous to determine the overall methylation status of a cell's entire genomic DNA using the methods of the invention. The overall methylation status of one cell may then be compared to that of another. For example, the overall (i.e., global) methylation status of a cell treated with a compound may be compared to that of a cell not treated with the compound. As used herein, by “compound” is meant an atom (e.g., arsenic or hydrogen), a molecule (e.g., oxygen or carbon dioxide), a chemical (e.g., phenobarbital), or a macromolecule, such as a protein, polysaccharide, or lipid. Moreover, a change in the methylation status of a cell may be an indication that the cell is cancerous, or is predisposed to becoming cancerous. Thus, in another example, the methylation status of a cell suspected of being cancerous may be compared to that of a normal cell. Note, that the methylation status of an individual nucleic acid molecule (e.g., a particular gene) may be compared between a cell treated with a compound and an untreated cell, and a cell suspected of being cancerous and a normal cell.

In a further aspect, the invention provides a method for determining the level of expression of a target nucleic acid molecule by a cell. The method of this aspect includes contacting the target double-stranded nucleic acid molecule isolated from the cell with a methylation-sensitive restriction endonuclease under conditions wherein the target double-stranded nucleic acid molecule is cleaved at a site recognized by the methylation-sensitive restriction endonuclease if the site is not methylated. Next, the target double-stranded nucleic acid molecule is PCR amplified with a detectably labeled primer that that hybridizes to a predetermined region of a strand of the double-stranded nucleic acid molecule, and the PCR product detected by capillary electrophoresis. The number of PCR products is inversely related to the level of expression of the target double-stranded nucleic acid molecule by the cell. In some embodiments, the detectably labeled primer is labeled with a fluorophore.

In some embodiments, the target double-stranded nucleic acid molecule is isolated from a cell, such as a mammalian cell. In some embodiments, the target double-stranded nucleic acid molecule is heterologous to the cell.

In situations where at least part of the sequence of the double-stranded nucleic acid molecule is known, the predetermined region of the strand of the double-stranded nucleic acid molecule to which the primer hybridizes may be a region on one of the strands of the nucleic acid molecule. For example, the detectably labeled primer can be designed to be complementary to (i.e., able to hybridize to) the 3′ end of one of the strands of the nucleic acid molecule.

In some embodiments, the double-stranded nucleic acid molecule contacted with the methylation-sensitive restriction endonuclease is PCR amplified with a detectably labeled primer that hybridizes to a predetermined region of a strand of the double-stranded nucleic acid molecule and a second primer that hybridizes to a second predetermined region of a second strand of the double-stranded nucleic acid molecule. Because the first primer is detectably labeled, the second primer need not be labeled.

In accordance with the invention, if the target double-stranded nucleic acid molecule shows a high level of methylation, then the cell from which that target double-stranded nucleic acid molecule is unlikely to express high levels of the protein encoded by the target double-stranded nucleic acid molecule. This result is particularly useful in the context of gene therapy. In one non-limiting example, patient suffering from adenosine deaminase (ADA) deficiency can be treated by reconstitution with white blood cells genetically engineered to express ADA. In other words, the white blood cells may be manipulated in vitro to express ADA, and those cells returned to the patient. However, not all the white blood cells introduced with a nucleic acid molecule encoding ADA (where the ADA is positioned for expression in the cell) will express equal amounts of ADA. White blood cells introduced with a nucleic acid molecule encoding ADA can be screened, not only for their ability to produce ADA protein, but also can be screened according to the methods of the invention to determine the amount of methylation of the introduced nucleic acid molecule. Those cells that do not show high levels of methylation of the introduced nucleic acid molecule are those that will likely continue to secrete high levels of ADA protein in the future. It is these cells that show low levels of methylation of the introduced nucleic acid molecule will be returned to the patient.

In some embodiments of the invention, some methylation of the introduced nucleic acid molecule may be desired. For example, a hemophiliac patient may lack cells that produce adequate amounts of a clotting factor (e.g., Factor VII). Hematopoietic stem cells from the patient's bone marrow (or cord blood, or from a blood relative) may be genetically engineered to express this clotting factor. However, it may be undesirable for the cells to express too much clotting factor (e.g., too much clotting factor may result in a higher propensity to develop stroke or atherosclerosis). Thus, an expression plasmid or vector containing the nucleic acid molecule encoding the clotting factor can be introduced into the hematopoietic stem cells, and those stem cells (1) screened for an ability to secrete the clotting factor and (2) screened, using the methods of the invention, for the level of methylation of the introduced nucleic acid molecule. Those cells secrete the clotting factor and that show a moderate amount of methylation of the introduced nucleic acid molecule (i.e., the nucleic acid molecule that encoding the clotting factor) will be returned to the patient.

In accordance with the invention, if the target double-stranded nucleic acid molecule is isolated form a cell suspected of being diseased and/or cancerous. Alterations in methylation status are found is diseased and/or cancerous cells (see, e.g., Castro et al., Clin. Chemistry 49(8): 1292-1296, 2003; Singh et al., Clin. Genet. 64: 451-460, 2003; Gama-Sosa et al., Nucleic Acids Res. 11: 6883-6894, 1983; Esteller et al., Cancer Res. 61: 3225-3229, 2001). The methylation status of the suspect cell (i.e., the cell suspected of being diseased ad/or cancerous) is determined according to the invention and compared to the methylation status of a normal cell. A change in the methylation status of the suspect cell as compared to the methylation status of the normal cell indicates that the suspect cell may, in fact, be diseased and/or cancerous. Subsequent tests (e.g., histology, expression of disease- or cancer-dependent genes) can also be employed to confirm that the suspect cell is diseased and/or cancerous.

In some embodiments, the predetermined region of the strand of the double-stranded nucleic acid molecule to which the primer hybridizes to is a GC-rich region in the nucleic acid molecule. Such a primer will arbitrarily amplify numerous nucleic acid molecules in a sample. For example, in some embodiments, the target double-stranded nucleic acid molecule is genomic DNA. Genomic DNA can be isolated from cells and the cells' methylation status determined using the methods of the invention.

In accordance with the invention, the methylation status of a cell contacted with a compound can be compared to the methylation status of a cell not contacted by the compound. In some embodiments, the number of PCR products resulting from the nucleic acid molecule digested with the methylation-sensitive endonuclease is greater than the number of PCR products resulting from the nucleic acid molecule digested with the methylation-insensitive endonuclease. The nucleic acid molecule may be genomic DNA (e.g., isolated from a cell that has been contacted with a compound).

With the increasing amount of information about key proteins that may be involved in the inception and/or propagation of a particular disease, the pharmaceutical industry is awash in candidate drugs that may confer beneficial results to patients. Using advanced technologies, such as combinatorial chemistry and high throughput screens, numerous potential lead compounds in very limited quantities are readily identified that can target any given key protein. The time and cost required to develop each of these compounds to the point of their use in clinical trials is considerable. And all too often, during clinical or preclinical trials, the compound is found to result in unwanted side effects, such as toxicity (Cockerell et al., Toxicol. Pathol. 30(1): 4-7, 2002).

Typically, initial assessments of toxicity include measurements of cytolethality and genotoxicity (including mutagenicity). Knowledge concerning the mutagenic potential of a compound is an important component of a basic, initial safety assessment (Ames et al., Mutat. Res. 62(2): 393-399, 1979; Rueff et al., Mutat. Res. 353(1-2): 151-176, 1996). However, different mutagenicity assays performed on the same compound can produce markedly disparate results (Choi et al., J. Tox. Environl. Health 49(3): 271-284, 1996). Structure-activity relationships often provide a basis for selection of potential drug candidates in the pharmaceutical industry, and this approach has also been used to try to identify compounds acting at sites known to elicit a toxic response (Woo et al., Toxicol. Letters 79: 219-228, 1995). Toxicogenomics holds out the potential to develop into a useful screening tool for identification of the toxic potential of chemicals (Tennant, Environ. Health Perspect. 110(1): A8-10, 2002). However, a substantial effort is necessary in order to evaluate this approach, including data analysis, more thoroughly before it can be employed on a routine basis. Furthermore, toxicogenomic analysis (e.g., measurement of changes in gene expression) does not provide insight regarding the possible mechanism(s) underlying any changes observed. On the contrary, evaluation of methylation status can provide insight regarding alteration/stability of a key mechanism responsible for regulating genes expression.

The methods of the invention are useful for quickly identifying those compounds that do not result in toxicity prior to the significant investment of time and/or money in developing a compound for administration to patients. The methods of the invention can serve as informative preliminary tests to predict the toxic potential of chemicals to prioritize them for further evaluation.

Thus, in a further aspect, the invention provides a method for determining if a compound affects the methylation status of a cell comprising isolating a double-stranded nucleic acid molecule from a cell contacted with the compound, and contacting that double-stranded nucleic acid molecule with a methylation-sensitive restriction endonuclease under conditions wherein the double-stranded nucleic acid molecule is cleaved at a site recognized by the methylation-sensitive restriction endonuclease if the site is not methylated. Next, the double-stranded nucleic acid molecule is PCR amplified with a detectably labeled primer that hybridizes to a predetermined region of a strand of the double-stranded nucleic acid molecule, and the PCR product detected using capillary electrophoresis. A double-stranded nucleic acid molecule isolated from a cell not contacted with the compound is also digested with the methylation-sensitive restriction endonuclease and PCR amplified, where the PCR product is detected using capillary electrophoresis. The PCR products from the two cells (i.e., the cell contacted with the compound and the cell not contacted with the compound) are compared, where a difference indicates that the compound affects the methylation status of the cell. In some embodiments, the detectably labeled primer is labeled with a fluorophore.

The nucleic acid molecule, in accordance with the invention, may be genomic DNA. Genomic DNA from a cell contacted with a compound is thus compared to genomic DNA from a cell not contacted with the compound. Where genomic DNA is analyzed, the overall pattern of the results can be compared. In particular embodiments, the predetermined region to which the primer hybridizes is a GC-rich region.

One particular target gene of a cell contacted with a compound may be analyzed with the methods of the invention. For example, the methods of the invention may be employed to assess the effect on cellular methylation of a compound suspected of being toxic to cells. For example, keratinocytes require expression of beta-1 integrin subunit to maintain their stem cell potential properties (see, e.g., Zhu et al., Proc. Natl. Acad. Sci. USA 96: 6728-6733, 1999; Carroll et al., Cell 83: 957-968, 1995). Thus, in accordance with the invention, the nucleic acid molecule encoding the beta-1 integrin subunit can be isolated from a keratinocyte that has been contacted with a compound, and the methylation status of the nucleic acid molecule determined and compared to that of a keratinocyte not contacted with the compound.

In some embodiments, the difference is an increase in the number of PCR products from the cell contacted with the compound as compared to the number of PCR products from the cell not contacted with the compound. Thus, the nucleic acid molecule from the compound treated cell is more heavily methylated than the nucleic acid from a cell not treated with the compound. The compound may alter methylation of a particular nucleic acid molecule (e.g., a particular gene) in a cell or may alter a cell's overall methylation status.

In some embodiments, the compound abrogates the growth of the cell. By “abrogates the growth” is meant that a cell contacted with a compound grows (i.e., divides or proliferates) at a rate slower than that cell if the cell were not contacted with the compound. Such a determination can be made by comparing a cell contacted with a compound with an uncontacted cell of similar lineage and phenotype (e.g., compare a contacted mouse liver cell with an uncontacted mouse liver cell). In some embodiments, the compound is toxic to the cell. By “toxic” means that a compound, when used to contact a cell, causes the death of that cell.

In one non-limiting example, the methods of the invention provide an initial assessment of a compound's toxic potential. For example, a cell (e.g., a mammalian cell) may be contacted with (e.g., by being cultured in the presence of) a compound, and the cytolethality of that compound (i.e., concentration of the compound that kills the cells) determined. A DNA methylation status determination using the methods of the invention could add additional valuable information. For example, if two lead compounds are tested, where one alters methylation at non-cytotoxic concentrations and the other, which does not, then the later compound is likely to be less toxic. This information could be valuable in the pharmaceutical industry where, at early stages of drug development, one is often dealing with small (mg amounts) of multiple compounds that seem to have promise. Thus, a methylation status determination may be very helpful regarding the providing of information (along with the standard cytolethality and capacity to be mutagenic) that could help prioritize these compounds based upon potential to cause toxicity (i.e., those that are less potentially toxic could be selected for further consideration). Thus, weeding out possibly toxic compounds at an early stage of development would save time and money.

The methods of the invention are also useful for identifying toxic compound in an environment where one of many different compounds may be toxic. For example, organic waste from a chemical plant may leak into the ground water, and cause sickness in the surrounding flora and fauna (including human). The different compounds in the waste may be used to contact cells, and the cells tested in accordance with the methods of the invention to identify which compound induces an alteration in the methylation status of the cell. In this situation, those compounds that induce alterations in methylation status may be selected for more thorough evaluation.

In some embodiments, the compound causes a decrease in the methylation of the cell's nucleic acid molecule (and thereby, a decrease in the number of PCR products). In other words, a decrease in the number of PCR products from the compound-contacted cell as compared to the cell not contacted with the compound is observed.

The compound may thereby enhance the growth of the cell. As used herein, “enhances the growth of a cell” means that a cell contacted with a compound grows (i.e., divides or proliferates) at a rate faster than such a cell not contacted with the compound. Such a determination can be made by comparing a contacted cell with an uncontacted cell of similar lineage and phenotype (e.g., compare a contacted human skin cell with an uncontacted human skin cell). In some embodiments, the compound may be a carcinogen. As used herein, by “carcinogen” means that a compound, when administered to an animal, causes cancer in that animal.

In a variation of this embodiment of the invention, the methylation status of tissue suspected of being cancerous (e.g., a mole or a lymph node) can be determined, to assess the possibility that the tissue is indeed cancerous (i.e., malignant). In one non-limiting example of this embodiment, tissue suspected of being cancerous (i.e., suspect tissue) and tissue adjacent to that suspect tissue that does not appear to be cancerous (i.e., normal tissue) can be biopsied, and DNA isolated from the biopsied tissue according to standard methods. The DNA is then isolated according to the methods of the invention, and differences in the methylation status between the two tissues compared. Abnormal patterns of DNA methylation are one of the most common molecular features observed in human and animal cancers (Gama-Sosa et al., Nucleic Acids Res. 11: 6883-6894, 1983; Esteller et al., Cancer Res. 61: 3225-3229, 2001). Altered methylation has been observed in, for example, human breast neoplasms (Miller et al., Cancer Res. 63: 7641-7645, 2003; Huynh et al., Cancer Res. 56: 4856-4870, 1996; DiNardo et al., Oncogene 20: 5331-5340, 2001; Krassenstein et al., Clin. Cancer Res. 10: 28-32, 2004), prostate carcinomas (Graff et al., Cancer Res. 55: 5195-5199), colorectal carcinoma (Hiltunen et al., Int. J. Cancer 70: 644-648, 1997; Cui et al., Cancer Res. 62: 6442-6446, 2002; Lengauer et al., Proc. Natl. Acad. Sci. 94: 2545-2550, 1997); gastric carinoma (Cravo et al., Gut 39: 434-438, 1996), cervical carinoma (Kim et al., Cancer 74: 893-899, 1994), pancreatic duct adenocarcinoma (Sato et al., Cancer Res. 63: 4158-4166, 2003), hepatocellular carinomas (Shen et al., Hepato-Gasteroenterology 45: 1753-1759, 1998; Lin et al., Cancer Res. 61: 4238-4243, 2001), and acute myelogenous and acute lymphocytic leukemias (Herman et al., Cancer Res. 57: 837-841, 1997). Therefore, the more altered the methylation pattern of the suspect tissue as compared to the normal tissue, the higher the possibility that the suspect tissue is cancerous (i.e., malignant). The extent to which methylation is altered may provide insight concerning high grade versus low grade malignancy and, therefore, be a prognostic as well as a diagnostic factor. Furthermore, new strategies aimed at reversing altered methylation in cancerous (or pre-cancerous) tissue may be developed in the future. In this case, it would be helpful to know which cancers, in particular patients, exhibit a high degree of altered methylation so that therapy could be targeted rationally to specific individuals. Additionally, there is interest in developing cancer chemotherapeutic drugs that act through a mechanism involving alteration of DNA methylation (one drug, azacytidine, that acts in this fashion is on the market currently). Therefore, the PCR-capillary electrophoresis procedure described here may be used effectively to test for chemicals that might be developed into drugs in this category.

In certain embodiments, the overall methylation status of the suspect tissue is lower than the overall methylation status of the normal tissue.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein. Such equivalents are considered to be within the scope of this invention, and are covered by the following claims.

### 6. EXAMPLES

6.1. Arbitrarily Primed PCR and Capillary Electrophoresis Separation and Detection

The method of the invention was performed to analyze the methylation status of genomic DNA from murine liver cells. For these studies, DNA was isolated from animal tissue or cells using TRIzol Reagent (commercially available from Invitrogen, Carlsbad, Calif.), according to the manufacturer's protocol. Other methods of DNA isolation such as standard phenol/chloroform are also acceptable.

For each DNA sample, of which duplicates or triplicates were prepared, two double digests with restriction enzymes were performed. One restriction enzyme (i.e., RsaI) that is not affected by methylation of its recognition sequence was used. RsaI is a methylation insensitive enzyme that recognizes the site 5′GTAC′ (cutting between the internal thymine and adenine residue). Digestion with this enzyme produced DNA of manageable size fragments.

The second restriction enzyme was affected by methylation of its recognition sequence (i.e., does not cut DNA if this sequence is methylated). In this example, one RsaI and MspI double digest and one RsaI and HpaII double digest was used. Both MspI and HpaII are methylation-sensitive enzymes that recognize 5′CCGG 3′ sites, and cut between the internal cytosine and guanine. However, MspI does not digest (i.e., will not cut or cleave) DNA if the external cytosine (i.e., the C at the 5′ position of the recognition site) is methylated, while HpaII does not restrict DNA if the internal cytosine (i.e., 5′CCGG 3′, where the underlined C residue is methylated) is methylated. Both MspI and HpaII will digest the 5′CCGG 3′ site if the site is unmethylated (Mann and Smith, Nuc. Acids Res. 4: 4238-4243, 1977).

Restriction digests contained 1 μg DNA and 5.0 units RsaI (Roche, Indianapolis, Ind.) in Roche Buffer L. Samples were incubated for 1 hour at 37° C. before the addition of 2.5 units of either MspI (Roche) or HpaII (Roche). A second 2.5 unit aliquot of the respective enzyme (i.e., either MspI or HpaII) was added after an additional 2 hours. Total incubation time was 18 hours. The enzymes were inactivated by incubating at 65° C. for 10 minutes. Samples were stored at 4° C. until needed.

PCR was performed on the restriction digests using a single 5′ fluorescently labeled arbitrary primer. The fluorescent label, hexachlorofluorescein (HEX™), was added to the 5′ end of the arbitrary primer. This labeled primer, 5′ HEX™-AACCCTCACCCTAACCCCGG 3′ (SEQ ID NO: ______) (custom made by Intergrated DNA Technologies; Coralville, Iowa) was designed to bind well to GC rich regions. All PCR reactions were set up in a sterile laminar flow hood on ice. Each reaction was composed of 5.0 μl of the restriction digest, 0.8 μM primer, 1.0 unit Taq polymerase (Invitrogen), 1× MasterAmp™ PCR PreMix L (commercially available from Epicentre®; Madison, Wis.), and glass distilled water (GDW) to volume. The Taq polymerase was added to the reaction following a 5 minute incubation at 80° C. Cycling conditions were as follows: 94° C. for 2 minutes, 5 cycles of 94° C. for 30 seconds, 40° C. for 1 minute, and 72° C. for 90 seconds, 40 cycles of 94° C. for 15 seconds, 55° C. for 15 seconds, and 72° C. for 1 minute, and a single time delay of 5 minutes at 72° C. followed by a 4° C. soak. The PCR samples (10-50 μl) were desalted and purified at the Genomics Technology Support Facility (GTSF) at Michigan State University using a Sephadex G50 superfine matrix. Other commercially available PCR purification columns such as QIAquick® PCR Purification Kit from Qiagen Inc. (Valencia, Calif.) or Microcon® Centrifugal Filter Devices from Millipore (Billerica, Mass.) would also achieve the necessary refinement.

8 ng (note that anywhere from approximately 4-10 ng could be used) of each purified and desalted PCR product are added to a mixture of formamide and a carboxy-X-rodamine (ROX™)-labeled 1000 bp size marker. Examples of additional fluorescent labels for the size marker include HEX™ and 6-FAM™. From this mixture, 2 μl was injected for electrophoresis using a 10 second injection time. Variations in volume injected and injection time are possible. This procedure was carried out using an Applied Biosystems 3700 Genetic Analyzer at GTSF, which is a fluorescence-based DNA analysis system. Sixteen capillaries, each 36 cm long and filled with a polymer, POP4, were run in parallel. Systems supporting greater than or fewer than 16 capillaries with 20-50 cm lengths can also be employed. The ROX-labeled 1000 bp size marker, which contains fragment sizes of 50, 75, 100, 125, 150, 200, 250, 300, 350, 400, 450, 475, 500, 550, 600, 650, 700, 750, 800, 850, 900, 950, and 1000 base pairs, was simultaneously run with each sample in order to accurately size the PCR products produced. The size marker also acted an internal control to ensure the run was carried out properly. All data were gathered using the program GeneScan3.7 (commercially available from Applied Biosystems, Foster City, Calif.) which compiles the results as size of PCR product in base pairs with a corresponding peak height representative of the amount of PCR product generated. Programs such as Genotyper and Genographer (which can be freely downloaded from websites hordeum.oscs.montana.edu/genographer/help/install.html and www.vgl.ucdavis.edu/informatics/STRand/download.html) would achieve the same data output. Only fragments greater than 100 bp in length and only peak heights areas with corresponding peak heights greater than 100 units were included to minimize incorporating primer dimmer peaks and background into the data analysis.

To analyze the data, each sample was aligned according to fragment sizes. Peak height area averages were calculated for the control group at each PCR fragment size for a particular digest, either the RsaI/MspI or RsaI/HpaII digest. To then compare changes between treated and control groups within a digest, each treated sample was calculated as a percent of the averaged control using the following equations:
% MspI control=((MspI treated−MspI averaged control)/MspI averaged control)×100
% HpaII control=((HpaII treated−HpaII averaged control)/HpaII averaged control)×100
These results are plotted using the Excel program (Microsoft) as size of fragment in base pairs versus percent averaged control. In this manner, all positives values indicate areas of hypermethylation while negative values represent areas of hypomethylation.

Table II below shows raw data from one control sample of mouse liver DNA that was digested with RsaI/MspI or RsaI/HpaII. Because the majority of currently identified 5-methylcytosine in eurkaoyotic cells occurs at cytosine residues immediately 5′ to guanine residues (i.e., 5′-CG-3′, where the underlined C is methylated), the enzyme MspI, which will not digest (i.e., cleave or cut) the DNA when the external cytosine is methylated within its recognition sequence of 5′CCGG 3′, should digest the DNA more thoroughly than HpaII. Furthermore, HpaII does not digest the DNA when the internal cytosine is methylated within the same recognition sequence of 5′CCGG3′. Comparison of peak area averages at each size fragment for each digest should reveal more restriction by MspI.

To test this hypothesis, the MspI and HpaII digests of one sample were compared (in a sense, this analysis is analogous to comparing treated and control samples). Raw data for a sample of mouse liver DNA with each digest run in triplicate are shown on Table II.

TABLE II

| | MspI Digest | | | HpaII Digest | | |
|---|---|---|---|---|---|---|
| | Replicate 1 | Replicate 2 | Replicate 3 | Replicate 1 | Replicate 2 | Replicate 3 |
| Base Pairs | Peak Area | | | | | |
| 164 | | 3070 | 1687 | 1703 | 1213 | 2052 |
| 192 | | | | 1830 | | 1811 |
| 200 | | 7047 | 2691 | 4879 | 4421 | 5986 |
| 270 | | 2805 | 2002 | 2998 | 2744 | 3716 |
| 271 | | 3839 | 2200 | 4073 | 3548 | 4749 |
| 276 | 2540 | 23696 | 10569 | 6182 | 5623 | 6653 |
| 287 | | | | 3889 | 3003 | 5799 |
| 290 | | 1863 | 1003 | 1367 | | 1270 |
| 307 | | 6409 | 1917 | 2321 | 1883 | 4759 |
| 311 | | | | 81356 | 72481 | 86896 |
| 312 | | | | 120666 | 93617 | 95484 |
| 315 | 9159 | 117852 | 62350 | 62523 | 52663 | 54173 |
| 323 | 8481 | 49798 | 30756 | 96005 | | 91420 |
| 324 | | | | 110449 | 176702 | 101642 |
| 358 | 28452 | 197295 | 122080 | 132276 | 139278 | 127167 |
| 360 | | | | 112215 | 116210 | 104002 |
| 365 | | | | 2559 | | 1721 |
| 368 | | 2732 | 1378 | 6714 | 3667 | 4847 |
| 370 | | 2787 | 1863 | 6013 | 3144 | 4680 |
| 379 | 5092 | 14823 | 10692 | 18666 | 12783 | 20993 |
| 391 | | 10115 | 7606 | 4151 | 3826 | 5998 |
| 403 | | 12485 | 6188 | 8207 | 4311 | 10021 |
| 437 | 3267 | 7699 | 6527 | 5989 | 3897 | 6831 |
| 441 | 8412 | 40643 | 27340 | 36952 | 38264 | 31793 |
| 445 | 14126 | 64375 | 45849 | 69203 | | 55938 |
| 461 | 4701 | 8334 | 6756 | 8150 | 4001 | 10704 |
| 477 | 3978 | 13256 | 9730 | 12426 | 5620 | 9317 |
| 497 | 23108 | 50820 | 48466 | 78958 | 41365 | 62488 |
| 512 | | | | 2418 | | 2575 |
| 534 | | | | 5322 | | 5386 |
| 554 | | 27277 | 9149 | 27825 | 7246 | 17404 |
| 566 | 3746 | 24686 | 10171 | 30642 | 13648 | 23630 |
| 578 | | 7797 | 3037 | 10812 | | 5977 |
| 581 | | 4975 | 2488 | | | |
| 599 | | 16396 | 11198 | 28789 | 10890 | 14966 |
| 668 | | 17393 | 7830 | | | |

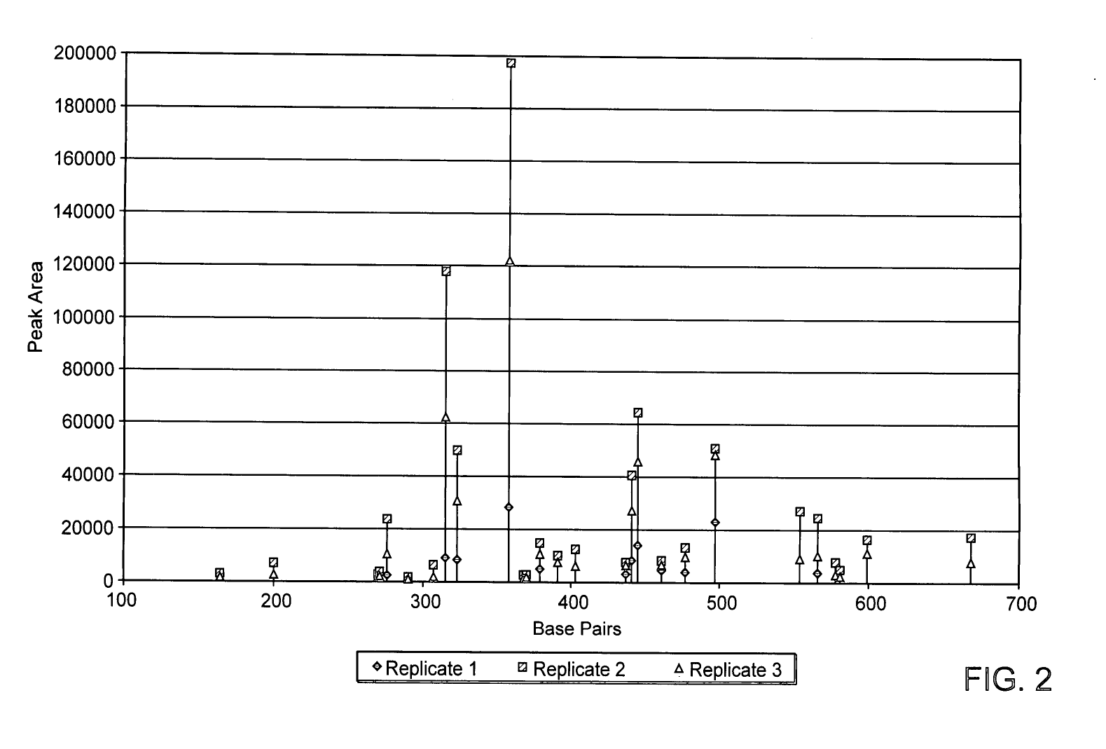

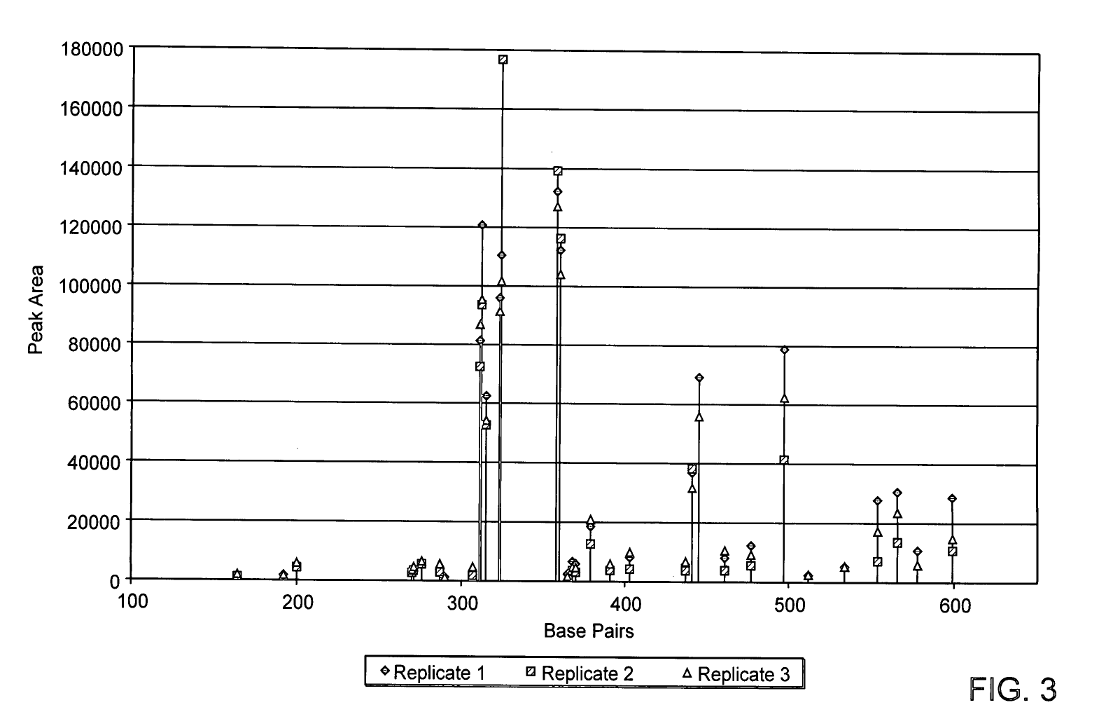

Only data points common to two or more replicates were included in Table II. FIG. 2 and FIG. 3 show the individual data points for each replicate of a digest plotted as size expressed as base pairs vs. peak area.

Calculations are shown in Table III.

TABLE III

| | RsaI/MspI Digest | | | | RsaI/HpaII Digest | | | | % | Opposite % |
|---|---|---|---|---|---|---|---|---|---|---|
| Base | Peak Area | | | | Peak Area | | | | Average | Average |
| Pairs | Replicate 1 | Replicate 2 | Replicate 3 | Average | Replicate 1 | Replicate 2 | Replicate 3 | Average | HpaII | HpaII* |
| 164 | | 3070 | 1687 | 2379 | 1703 | 1213 | 2052 | 1656 | 43.6 | −43.6 |
| 192 | | | | | 1830 | | 1811 | 1821 | −100.0 | 100.0 |
| 200 | | 7047 | 2691 | 4869 | 4879 | 4421 | 5986 | 5095 | −4.4 | 4.4 |
| 270 | | 2805 | 2002 | 2404 | 2998 | 2744 | 3716 | 3153 | −23.8 | 23.8 |
| 271 | | 3839 | 2200 | 3020 | 4073 | 3548 | 4749 | 4123 | −26.8 | 26.8 |
| 276 | 2540 | 23696 | 10569 | 12268 | 6182 | 5623 | 6653 | 6153 | 99.4 | −99.4 |
| 287 | | | | | 3889 | 3003 | 5799 | 4230 | −100.0 | 100.0 |
| 290 | | 1863 | 1003 | 1433 | 1367 | | 1270 | 1319 | 8.7 | −8.7 |
| 307 | | 6409 | 1917 | 4163 | 2321 | 1883 | 4759 | 2988 | 39.3 | −39.3 |
| 311 | | | | | 81356 | 72481 | 86896 | 80244 | −100.0 | 100.0 |
| 312 | | | | | 120666 | 93617 | 95484 | 103256 | −100.0 | 100.0 |
| 315 | 9159 | 117852 | 62350 | 63120 | 62523 | 52663 | 54173 | 56453 | 11.8 | −11.8 |
| 323 | 8481 | 49798 | 30756 | 29678 | 96005 | | 91420 | 93713 | −68.3 | 68.3 |
| 324 | | | | | 110449 | 176702 | 101642 | 129598 | −100.0 | 100.0 |
| 358 | 28452 | 197295 | 122080 | 115942 | 132276 | 139278 | 127167 | 132907 | −12.8 | 12.8 |
| 360 | | | | | 112215 | 116210 | 104002 | 110809 | −100.0 | 100.0 |
| 365 | | | | | 2559 | | 1721 | 2140 | −100.0 | 100.0 |
| 368 | | 2732 | 1378 | 2055 | 6714 | 3667 | 4847 | 5076 | −59.5 | 59.5 |
| 370 | | 2787 | 1863 | 2325 | 6013 | 3144 | 4680 | 4612 | −49.6 | 49.6 |
| 379 | 5092 | 14823 | 10692 | 10202 | 18666 | 12783 | 20993 | 17481 | −41.6 | 41.6 |
| 391 | | 10115 | 7606 | 8861 | 4151 | 3826 | 5998 | 4658 | 90.2 | −90.2 |
| 403 | | 12485 | 6188 | 9337 | 8207 | 4311 | 10021 | 7513 | 24.3 | −24.3 |
| 437 | 3267 | 7699 | 6527 | 5831 | 5989 | 3897 | 6831 | 5572 | 4.6 | −4.6 |
| 441 | 8412 | 40643 | 27340 | 25465 | 36952 | 38264 | 31793 | 35670 | −28.6 | 28.6 |
| 445 | 14126 | 64375 | 45849 | 41450 | 69203 | | 55938 | 62571 | −33.8 | 33.8 |
| 461 | 4701 | 8334 | 6756 | 6597 | 8150 | 4001 | 10704 | 7618 | −13.4 | 13.4 |
| 477 | 3978 | 13256 | 9730 | 8988 | 12426 | 5620 | 9317 | 9121 | −1.5 | 1.5 |
| 497 | 23108 | 50820 | 48466 | 40798 | 78958 | 41365 | 62488 | 60937 | −33.0 | 33.0 |
| 512 | | | | | 2418 | | 2575 | 2497 | −100.0 | 100.0 |
| 534 | | | | | 5322 | | 5386 | 5354 | −100.0 | 100.0 |
| 554 | | 27277 | 9149 | 18213 | 27825 | 7246 | 17404 | 17492 | 4.1 | −4.1 |
| 566 | 3746 | 24686 | 10171 | 12868 | 30642 | 13648 | 23630 | 22640 | −43.2 | 43.2 |
| 578 | | 7797 | 3037 | 5417 | 10812 | | 5977 | 8395 | −35.5 | 35.5 |
| 581 | | 4975 | 2488 | 3732 | | | | | | |
| 599 | | 16396 | 11198 | 13797 | 28789 | 10890 | 14966 | 18215 | −24.3 | 24.3 |
| 668 | | 17393 | 7830 | 12612 | | | | | | |

For visualization of the data using this Excel program, the error bar function (note: this does not represent error of the data) was used to create the vertical lines representative of the values. In this manner a positive value had to be accompanied by its opposite value in order for the plot to display a negative error bar. Opposite values were listed for graphing purposes in order to create vertical lines representative of the values of the data.

Averages were calculated for each digest for every fragment size. To compare the MspI digest to the HpaII digest, the MspI digest was calculated as a percent of the HpaII digest using the following equation:
% average HpaII=((Average MspI−Average HpaII)/Average HpaII)×100.

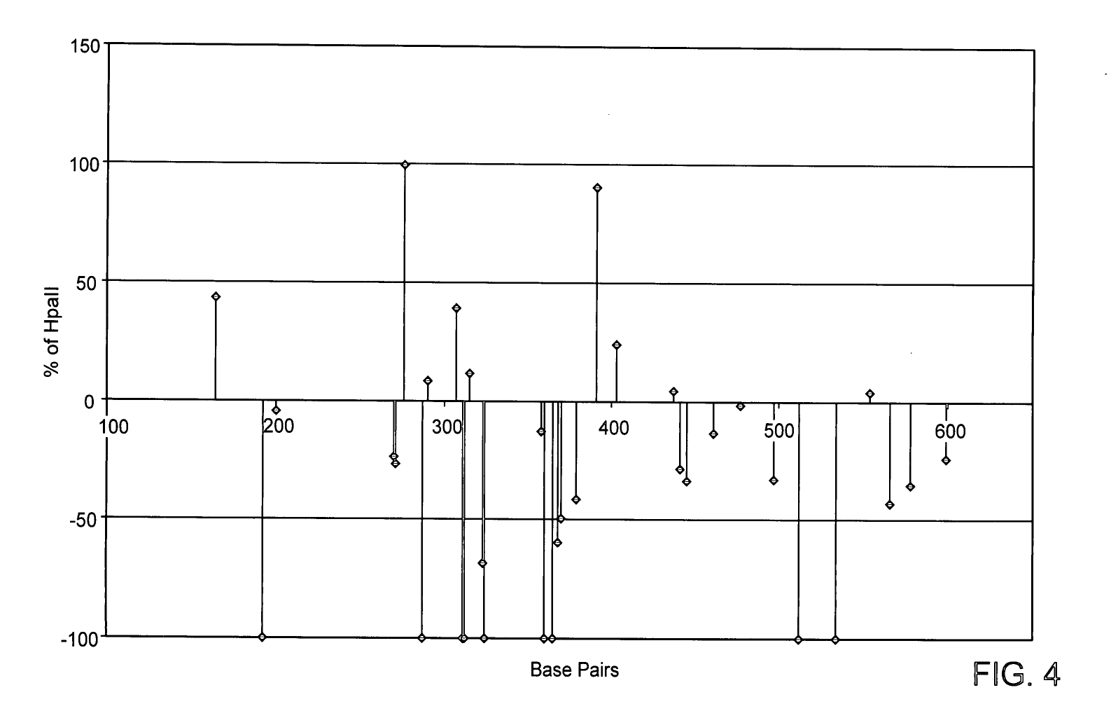

FIG. 4 shows the data of average percentage of PCR products formed when DNA was digested with RsaI and HpaII prior to PCR as compared with the average percentage of PCR products formed when DNA was digested with RsaI and MspI prior to PCR plotted as size in base pairs vs. percent of the averaged HpaII. The data are presented as average MspI−Average HpaII)/Average HpaII)×100. Thus, the positive values shown on the graph indicate less cutting by MspI than HpaII, while negative values represented more cutting by MspI than HpaII.

Both MspI and HpaII will cleave DNA at 5′CCGG-3′ sites if the DNA is not methylated. However, if the internal “C” is methylated, HpaII will not cleave while MspI will. Under control conditions, methylation occurs frequently at the internal “C” of 5′-CCGG-3′ sites. Therefore, MspI is expected to cleave DNA more frequently than HpaII. With 9 positive values and 25 negative values, this comparison shows, as expected, a greater amount of restriction by MspI than HpaII. Thus, the hypothesis that more of the cells' genomic DNA had CpG methylation (where the underlined C is methylated) than CpCpG methylation (where the underlined C is methylated) was demonstrated.

6.2. Use of Methylation Status to For Toxicity Assessment

H4IIE rat hepatoma cells (between passages 7-9) are grown in 96- and 6-well plates for in vitro toxicity analysis and for methylation analysis, respectively. Results from these in vitro toxicity assessments do not vary between 96 and 6 well plates. Cells to be used for methylation analysis are dosed with concentrations of compounds deemed to be cytolethal and non-cytolethal based on a battery of in vitro cytolethality assessments. After a 72 hour incubation, cells are washed twice with PBS, trypsinized, centrifuged, and frozen at −80° C. until use. DNA is extracted using Trizol® reagent (Sigma-Aldrich®, St. Louis, Mo.) and stored at 4° C. until use.

The compound 5-aza-2′deoxycytidine (dAzaC; commercially available from Sigma Aldrich®, St. Louis, Mo.), is a cytosine analog known to cause demethylation by incorporating into DNA and irreversibly binding DNA methyltransferase, thus inhibiting methylation of newly replicated DNA (Lu and Randerath, Mol. Pharmacol. 26(3): 594-603, 1984).

In addition, four model compounds with varying modes of action and different toxic effects are used. None of these compounds is known to have any effect on DNA methylation. Camptothecin is an S-phase specific anticancer agent that inhibits the activity of DNA topoisomerase I, leading to replication fork arrest as well as single- and double-strand DNA breaks (Morris and Geller, J. Cell Biol. 134(3): 757-770, 1996). 5-fluorouracil (5-FU) is a pyrimidine analog that is metabolized to 5-fluorodeoxyrudine monophosphate, a compound that competes with deoxyuridine monophosplate for thymidylate synthetase. Normally, thymidylate synthetase catalyzes the conversion of deoxyuridine monophosphate to thymidine monophosphate, a precursor of thymidine triphosphate, and a necessary component of DNA (Parker and Cheng, Pharmacol. Ther. 48(3): 381-395, 1990). Thus, the overall effect of 5-FU is to inhibit replication. Rotenone inhibits complex I of the mitochondrial oxidative phosphorylation chain, stopping the supply of electrons to quinol cytochrome c oxidoreductase. This decreases ATP production and the release of cytochrome c from the mitochondria as well as the increased permeability of the mitochondrial membrane leads to caspase-mediated apoptosis (Pei et al., FASEB J. 17(3): 520-522, 2003). Staurosporine is a nonspecific inhibitor of protein kinases which promotes apoptosis through both caspase-dependent and independent mechanisms (Belmokhtar et al., Oncogene 20: 3354-3362, 2001). Staurosporine also inhibits the catalytic activity of topoisomerase II by blocking the transfer of phosphodiester bonds from DNA to the active tyrosine site (Lassota et al., J. Biol. Chem. 271(42): 26418-26423, 1996). All compounds described were purchased from Sigma-Aldrich® (St. Louis, Mo.).

In vitro toxicity assessments for each compound will include measurements of adenosine triphosphate (ATP), cell number, glutathione-S-transferase (GST), and 3-(4,5-dimethylthiazol-2-yl)-2,5-diphenyltetrazolium bromide (MTT) as part of the Tox Cluster battery of assays described by McKim et al. (Toxicol. Sci. Suppl. 60: 306, 2001).

ATP Assay

ATP serves as the principal immediate donor of free energy and is present in all metabolically active cells (Crouch et al., J. Immunol. Methods, 160: 81-88, 1993). Levels of ATP decline rapidly when cells are injured, and this is measured using an ATP bioluminescence assay in which a luciferin ATP substrate is added which interacts with ATP and oxygen to form oxyluciferin, AMP, PPᵢ, CO₂, and light (Crouch et al., supra). The ATPLite-M™ Packard ATP bioluminescence assay kit is used to measure the amount of ATP in the H4IIE cells. The amount of ATP is extrapolated from the amount of light emitted as measured by a spectrophotometer (Packard®, Palo Alto, Calif.). Results from this assay are expressed as percentage of control values.

Cellular Proliferation Assay

Measurements of cellular proliferation provide a general measure of toxicity. Cell number can be assessed using the CyQUANT cell proliferation assay kit from Molecular Probes® (Eugene, Oreg.), a highly sensitive, fluorescence based microplate assay for determining the number of cultured cells (Jones et al., J. Immunol. Methods 254: 85-98, 2001). For this cell proliferation method, cells are rinsed with PBS to remove dead cells no longer adhering to the plate, lysed, and the DNA is stained using the CyQUANT fluorescent dye. Fluorescence is measured using a Packard Spectracount® fluorescence reader. Using a standard curve that can be generated from the fluorescence readings of known amounts of H4IIE cells, the cell number is extrapolated.

GST Assay

GST leakage is linked to a loss of membrane integrity and necrosis in hepatocytes, and thus, the amount of GST is related to cell viability (Giannini et al., Clin. Biochem. 33(4): 297-301, 2000). To measure GST release into the serum, the Biotrin® (Czech republic) Rat Alpha GST Enzyme Immunoassay is used. After 72 hours, serum from the cells is removed, is diluted 1:4 with media, and 100 μl/well of the diluted serum is placed into 96-well plates coated with IgG antibody. The cells are incubated for 1 hour at room temperature using a rotary mixer. Plates are then washed 6 times using the Biotrin Wash Buffer. After removing all the fluid from the plate, 100 μl/well of the Biotrin Conjugate is added. This conjugate binds to the IgG-bound GST. Plates are incubated with the conjugate for 1 hour at room temperature using a rotary mixer, and then are washed 6 times using Biotrin Wash buffer. After removing all the fluid from the plate, 100 μl of Biotrin TMB substrate is added to each well. The plates are incubated for 15 min. at room temperature using a rotary mixer. Following incubation, 50 μl stop solution is added to each well and plates are read using a Packard Spectracount spectrophotomer. The % damaged GST-releasing cells and % non-damaged cells (not releasing GST above basal values) is determined using a standard curve generated from standards containing known percentages of control and 50 μM digitonin-treated cells. Digitonin damages cells and elicits GST release. GST Results are presented as the % of control cells not releasing GST above basal values.

MTT Assay

MTT analysis provides a general measurement of mitochondrial dehydrogenase activity and cell viability (Rodriguez and Acosta, Toxicol. 117: 121-131, 1997). The MTT assay is based on the reduction of the soluble yellow MTT tetrazolium salt to a blue MTT formazan product by mitochondrial dehydrogenases (Mossman, J. Immunol. Methods 65: 55-63, 1983). Each well of H4IIE cells within 96-well plates can be incubated with 100 μl of a 0.5 mg/ml MTT solution for 3 hour. Following the MTT incubation, the media is removed by aspiration and 200 μl of isopropanol is added to each well to dissolve and solubilize the intracellular MTT formazan product. After a 20 min incubation with isopropanol (with shaking) in the dark, the optical density of each well can be assessed at 570 and 850 nm using a Packard Spectracount spectrophotometer. Results can be expressed as a percentage of control values. MTT is commercially available from Sigma-Aldrich®.

Next, cytolethal and non-cytolethal concentrations of compounds are selected. Based upon dose-response analysis, the threshold concentration is estimated to be the first concentration below which there was no statistically significant change compared to measurements in untreated control cells and above which there is a significant change in at least two of the parameters. A concentration equal to 10-25% of this value is used as the non-cytolethal concentration. The cytolethal concentration is selected as the first concentration at which the percent control values for at least two of the assays is between 25 and 40%. Thus, non-cytolethal and cytolethal concentrations are chosen in a uniform manner for each model compound. Additionally, these parameters are used to select non-cytolethal concentrations of dAzaC.

Global DNA methylation can be assessed using an SssI methylase assay. SssI methylase utilizes S-adenosyl methionine as a methyl group donor to methylate the 5′ position of cytosine at unmethylated CpG sites in DNA. Thus, the level of global DNA methylation can be determined by the amount of tritiated methyl groups from [³H—CH₃] S-adenosyl-L-methionine incorporated into DNA, since there is an inverse relationship between incorporation of radioactivity and the original degree of methylation (Balaghi and Wagner, Biochem. Biophys. Res. Commun. 193: 1184-1190, 1993). DNA (e.g., 1 μg) is incubated with 2 μCi [³H—CH₃] S-adenosyl-L-methionine (New England Nuclear, Boston, Mass.) and 3 units of SssI methylase (New England Biolabs, Beverly, Mass.) for 1 hour at 30° C. Results can be presented as counts per minute per microgram (cpm/μg) DNA. Numerous replicates (e.g., five) can be performed per sample. Graphical presentation can be performed using the Excel® program (Microsoft). Statistical analysis can be performed with Excel using two-tailed t-tests to compare the average cpm/μg DNA measurements between treatment groups and controls. A p value of <0.05 will be considered statistically significant.

Methylation analysis of GC-rich regions is next determined.

Restriction Digests

For each DNA sample, 3 restriction digests are performed as follows: RsaI alone, RsaI and MspI, and RsaI and HpaI. RsaI is a methylation-insensitive enzyme which will be used to cut (i.e., cleave or digest) the DNA into smaller fragments. As described in Example I, both MspI and HpaII are methylation-sensitive enzymes that cut between cytosine residues at 5′-CCGG-3′ sites. MspI will not cut if the external cytosine is methylated, and HpaII will not cut if the internal cytosine is methylated. Both MspI and HpaII will cut if the site is unmethylated (Mann and Smith, Nuc. Acids Res. 4: 4238-4243, 1977). All enzymes are commercially available from Roche® (Indianapolis, Ind.). Restriction digests are performed with 1 μg of DNA and 5.0 units of RsaI in Roche buffer L. After a 1 hour incubation (with shaking) in a water bath at 37° C., two 2.5 unit aliquots of MspI or HpaII are added, 2 hours apart. The total incubation time is 18 hour. The enzymes are inactivated by a 10 minute incubation at 65° C., and the digests were stored at 4° C. until amplified by PCR.

Arbitrarily Primed (AP)-[³³P] PCR

PCR are performed on restriction digests using a single primer that arbitrarily binds within GC-rich regions of DNA (Gonzalgo et al., Cancer. Res. 57: 594-599, 1997). Non-limiting primers that can be employed include:

| | |
|---|---|
| 5′-AACCCTCACCCTAACCCCGG-3′ | (SEQ ID NO:_) |
| 5′ TAACTCCATCCAACCCGGG 3′ | (SEQ ID NO:_) |
| 5′ AACCCCTAATCCCGGG 3′ | (SEQ ID NO:_) |
| 5′ ACCTCCCAATGCGC 3′ | (SEQ ID NO:_) |
| 5′ CATTCTACCCCATGCGC 3′ | (SEQ ID NO:_) |

The primer used has attached to its 5′ end any fluorescent label suitable for a capillary electrophoresis instrument. Non-limiting fluorescent labels include HEX, 6-FAM, JOE NHS Ester, and ROX™ NHS Ester. These, and other suitable labels are commercially available from Integrated DNA Technologies, Inc (Coralville, Iowa) or from Synthegen, LLC (Houston, Tex.). Note a primers with a label attached to their 5′ ends, can be ordered directly from Integrated DNA Technologies or Synthegen (i.e., these companies will synthesize the primer, with a sequence as requested, and attach the desired label to the primer's 5′ end).

Reactions are composed of 5 μl of the restriction digest (containing 1 μg digested DNA), 0.4 μM each primer, 1.25 units of Taq polymerase (Gibco BRL, Rockville, Md.), 1.5 mM MgCl₂, 60 mM Tris, 15 mM ammonium sulfate, 1.65 μCi α-[³³P]-dATP (New England Nuclear, Boston, Mass.), and glass-distilled water to volume. Samples are heated for 5 min at 94° C. before addition of dNTPs in order to minimize the possibility of primer-dimer formation. Cycling conditions included a single denature cycle for 2 minutes at 94° C., followed by 5 cycles under the following conditions: 30 seconds at 94° C., 1 minute at 40° C., 1.5 minutes at 72° C.; then 30 cycles of 94° C. for 30 seconds, 55° C. for 15 seconds, and 72° C. for 1 minutes, a time delay cycle for 5 minutes at 72° C., and a soak cycle at 4° C.

The PCR samples are desalted and purified at the Genomics Technology Support Facility (GTSF) at Michigan State University using a Sephadex G50 superfine matrix. Other commercially available PCR purification columns such as QIAquick® PCR Purification Kit from Qiagen Inc. (Valencia, Calif.) or Microcon® Centrifugal Filter Devices from Millipore (Billerica, Mass.) may also be used to desalt and purify the PCR samples.

At least 3 or 4 nanograms of each purified and desalted PCR product are added to a mixture of formamide and a fluorescent labeled 1000 bp size marker. The marker can be labeled with any fluorescent label other than the label used on the primer (e.g., if the primer is labeled with HEX™, the size marker can be labeled with any label except HEX™). The samples are analyzed using an Applied Biosystems 3700 Genetic Analyzer at GTSF, which is a fluorescence-based DNA analysis system. The ROX-labeled 1000 bp size marker was simultaneously run with the samples for sizing and normalization of the results. All data were gathered using the program GeneScan3.7 (commercially available from Applied Biosystems, Foster City, Calif.) which compiles the results as size of PCR product in base pairs with a corresponding peak area representative of the amount of PCR product generated. Programs such as Genotyper and Genographer (which can be freely downloaded from websites hordeum.oscs.montana.edu/genographer/help/install.html and www.vgl.ucdavis.edu/informatics/STRand/download.html) would achieve the same data output. Only fragments greater than 100 bp in length and only peak areas with corresponding peak heights greater than 100 units were included to minimize incorporating primer dimmer peaks and background into the data analysis.

To analyze the data, each sample is aligned according to fragment sizes. Peak area averages are calculated for the control group at each PCR fragment size for a particular digest, either the RsaI/MspI or RsaI/HpaII digest. To then compare changes between treated and control groups within a digest, each treated sample is calculated as a percent of the averaged control using the following equations:
% MspI control=((MspI treated−MspI averaged control)/MspI averaged control)×100
% HpaII control=((HpaII treated−HpaII averaged control)/HpaII averaged control)×100
These results are plotted using the Excel program (Microsoft) as size of fragment in base pairs versus percent averaged control. In this manner, all positives values indicate areas of hypermethylation while negative values represent areas of hypomethylation.

The results are predicted to show that cells treated with cytolethal concentrations of 5-FU or staurosporine show a decrease in global DNA methylation status. At non-cytolethal concentrations, the results are predicted to show that cells treated with dAzaC or staurosporine show a decrease in global DNA methylation status. Moreover, when a decrease in global DNA methylation status is observed, it is predicted that the number of CpG methylation increases (i.e., that more cytosine residues are methylated), where cytosine residue is 5′ of a guanine residue).

6.3. Methylation Status of Genomic DNA of Mice Treated with Phenobarbital

The method of the invention was performed to analyze the methylation status of genomic DNA from liver cells of mice treated with phenobarbital. Phenobarbital is known to alter the methylation status of DNA in murine liver (see, e.g., Ray et al., Molecular Carcinogenesis 9: 155-166, 1994; Counts et al., Carcinogenesis 17(6): 1251-1257, 1996; Watson et al., Toxicol. Sci. 68(1): 51-58, 2002).

Animals:

Male C57BL/6 mice were obtained from Charles River Laboratories and housed in a temperature-controlled environment with food and water given ad libitum. Five treatment animals were given a tumor-promoting dose of phenobarbital (PB; commercially available from Sigma Aldrich, St. Louis, Mo.) at a concentration of 0.05% (w/v) in the drinking water for 2 weeks. Five control animals were given the standard food and water diet. All animals were sacrificed by CO₂ asphyxiation and the livers were snap frozen at −80° C. DNA was isolated using TRIzol reagent (Invitrogen).

Restriction Digestion:

For each DNA sample (5 control DNA samples and 5 treated DNA samples), of which duplicates were prepared, one double digest (i.e., digestion with two different restriction enzymes) was performed. One restriction enzyme of the two used in the double digest was not affected by methylation of its recognition sequence and was used to cut the DNA into manageable size fragments. The other restriction enzyme in the double digest is affected by methylation of its recognition sequence (i.e., will not cut DNA if this sequence is methylated). For this study, a double digest with RsaI and HpaII was employed, where RsaI is a methylation insensitive enzyme and HpaII is a methylation sensitive enzyme. HpaII recognizes 5′CCGG 3′ sites, and cuts between the internal cytosine and guanine, but will not cut DNA if the internal cytosine is methylated. Restriction digests contained 1 μg DNA and 5.0 units RsaI (Roche) in Roche Buffer L. Samples were incubated for 1 hour at 37° C. before addition of 2.5 units HpaII (Roche). A second 2.5 unit aliquot of HpaII was added after an additional 2 hours. Total incubation time was 18 hrs. The enzymes were inactivated by incubating at 65° C. for 10 minutes. Samples were stored at 4° C. until needed.

Arbitrarily Primed PCR:

PCR was performed on restriction digests using a single arbitrary primer having the sequence 5′ AAC CCT CAC CCT AAC CCC GG 3′ (SEQ ID NO: ______). This primer was designed to bind well to GC-rich regions and the 5′CCGG 3′ sequence at its 3′ end increases the probability of primer annealing to the MspI and HpaII restriction site. This allows for the detection of methylation at the site of primer annealing and between sites of primer annealing. The fluorescent label, hexachlorofluorescein (HEX™), was added to the 5′ end of the arbitrary primer, which allows for the detection of PCR products via capillary electrophoresis. All PCR reactions were set up in a sterile laminar flow hood on ice. Each reaction was composed of 5.0 μl of the restriction digest, 0.8 μM primer, 1.0 unit Taq polymerase (Invitrogen), 1× MasterAmp™ PCR PreMix L (Epicentre®; Madison, Wis.), and glass distilled water (GDW) to volume. The Taq polymerase was added to the reaction following a 5 minute incubation at 80° C. Cycling conditions were as follows: 94° C. for 2 minute, 5 cycles of 94° C. for 30 seconds, 40° C. for 1 minute, and 72° C. for 1 minute 30 seconds, 40 cycles of 94° C. for 15 seconds, 55° C. for 15 seconds, and 72° C. for 1 minute, and a single time delay of 5 minutes at 72° C. followed by a 4° C. soak. The PCR samples were desalted and purified at the Genomics Technology Support Facility (GTSF) at Michigan State University using a sephadex G50 superfine matrix.

Capillary Electrophoresis Separation and Detection:

Eight nanograms of each purified and desalted PCR product was added to a mixture of formamide and a carboxy-X-rodamine (ROX™)-labeled 1000 bp size marker. From this mixture, 2 μl was injected for electrophoresis using a 10 second injection time. This procedure was carried out using an Applied Biosystems 3700 Genetic Analyzer at GTSF which is a fluorescence-based DNA analysis system. Sixteen capillaries, each 36 cm long and filled with a polymer, POP4, were run in parallel. The ROX-labeled 1000 bp size marker, which contains fragment sizes of 50, 75, 100, 125, 150, 200, 250, 300, 350, 400, 450, 475, 500, 550, 600, 650, 700, 750, 800, 850, 900, 950, and 1000 base pairs, was simultaneously run with each sample in order to accurately size the PCR products produced. The size marker also acted an internal control to ensure the run was carried out properly.

Capillary Electrophoresis Separation and Detection Data Analysis:

All data were gathered using the program GeneScan3.7 which compiles the results as size of PCR product in base pairs with a corresponding peak area representative of the amount of PCR product generated. Only fragments>100 bp and only peak areas with corresponding peak heights>100 units were included to minimize incorporating primer dimmer peaks and background into the data analysis.

Data was then compared across all the samples in terms of peak areas for each PCR product size. For example, all replicates reporting a peak area at 101 bp were compared. In order to more appropriately align the data for analysis, peak areas which spanned up to 3 base pairs were grouped together at a common size (e.g., a peak spanning 101 bp, 102 bp, and 103 bp was considered to be the same PCR product size). In the above example, the data would be grouped at 101, 102, or 103 base pairs depending on the size at which the most animals reported a peak area. Normally, peak areas only exhibited a 1 or 2 base pair size difference. At this point none of samples were averaged; they were just arranged according to size.

With the raw data in this ‘aligned’ version, base pair and peak area data points which were not reported in at least 50% of the number of animals and at least 50% of the total number of replicates were discarded. For 5 animals (either control or treated), each with 2 replicates, 3 of the 5 animals and 5 of the 10 replicates had to be reporting a peak area for the given PCR product size, otherwise the points were discarded.

Tables IV and V below shows raw data from control samples (Table IV) and Phenobarbital-treated samples (Table V) of mouse liver DNA that was double digested with RsaI/HpaII.

TABLE IV

| | Control Animals: RsaI/HpaII Digest | | | | | | | | | |
|---|---|---|---|---|---|---|---|---|---|---|
| Base | Animal 1 | | Animal 2 | | Animal 3 | | Animal 4 | | Animal 5 | |
| Pairs | Replicate 1 | Replicate 2 | Replicate 1 | Replicate 2 | Replicate 1 | Replicate 2 | Replicate 1 | Replicate 2 | Replicate 1 | Replicate 2 |
| 101 | 1424 | 1310 | 1125 | | | | 1092 | | 1677 | 1474 |
| 107 | 3390 | 1697 | | 1340 | | | | 1869 | 2442 | 2603 |
| 114 | | | | 3428 | 2141 | 2485 | | | 2036 | 1180 |
| 146 | | | | | | | | | | |
| 147 | | | | | | | | | | |
| 148 | | | | | | | | | | |
| 163 | 2737 | 5226 | 8821 | 3748 | 1572 | 4659 | 12232 | 3186 | 23663 | 5850 |
| 173 | | | | | | | | | | |
| 185 | | | | | | | | | | |
| 191 | 2619 | 4433 | 7204 | 1120 | | 4929 | 13665 | 3116 | 25892 | 4976 |
| 201 | | 8925 | 19765 | 54091 | 29766 | 13164 | 12585 | 5469 | 9159 | 15655 |
| 208 | | | 3444 | 8358 | 4668 | 1378 | | | 2808 | 2489 |
| 210 | | | 2509 | 4469 | 2531 | | 4395 | | 4036 | 4163 |
| 213 | | | 1740 | 3756 | 7798 | 8573 | 7713 | 3135 | 7110 | 13492 |
| 221 | | | 1751 | 4119 | | 757 | 1706 | | 5917 | |
| 238 | | | | | | | | | | |
| 244 | | 1593 | 3691 | 2622 | 4153 | | | | | 3008 |
| 246 | | | | | | | | | | |
| 252 | | | | | | | | | | |
| 271 | | 1715 | 2439 | 4503 | 3346 | | | | 2722 | |
| 273 | | 4403 | 6728 | 13679 | 8019 | 2192 | | | 4268 | |
| 275 | 5557 | 4914 | 3176 | | 4017 | 1466 | | 2054 | | |
| 277 | 3657 | 3515 | 15082 | 14114 | 16405 | 2989 | 4709 | | 5230 | 68073 |
| 278 | 6574 | 2557 | | 23753 | | 5179 | | 3891 | | |
| 279 | | 6829 | 14468 | 30952 | 15167 | | 7489 | | | |
| 284 | | 1168 | 1320 | 2735 | 2162 | | 3611 | | | |
| 287 | | | | | | | | | | |
| 289 | | 2043 | 2352 | 2493 | | 2398 | 5372 | | | 1682 |
| 295 | | | | | | | | | | |
| 315 | 234726 | 272009 | 443103 | | 447629 | 76001 | | | 263955 | 259714 |
| 325 | 72931 | 236090 | 285483 | | 250149 | 39717 | 28490 | 7448 | 204607 | 161799 |
| 342 | | | | | | | | | | |
| 358 | | | 231304 | 215788 | 231728 | | 247903 | 18136 | 174523 | 183754 |
| 362 | | 461083 | 335995 | 417314 | 366951 | 385871 | | | 222811 | 255884 |
| 381 | | 4709 | 4739 | 2840 | 1930 | 3769 | | | 20240 | |
| 404 | | | | | | | | | | |
| 406 | | | | | | | | | | |
| 408 | | 2513 | | 7008 | | 7668 | | | 8191 | |
| 445 | | 92205 | 76685 | 176000 | 81906 | 70065 | 14400 | | 157588 | 87673 |
| 479 | 3774 | | | | 3241 | 30061 | | | 33796 | 35143 |
| 498 | 23458 | 48136 | 11423 | 64388 | 26837 | 55271 | 14249 | | 68767 | 42114 |
| 513 | 4636 | | | | | 1138 | 4031 | | 3967 | 6816 |
| 555 | | | | | 8908 | 105982 | 7679 | 5164 | 128650 | |
| 566 | 15771 | 6029 | 3501 | 5859 | 10563 | 25709 | 10921 | | 52701 | 36664 |
| 580 | | | | | | 11992 | 3376 | | 29704 | 4329 |
| 600 | | 2886 | 3507 | 5678 | 4165 | 11380 | 5879 | | 17978 | |
| 602 | | | 5576 | 4285 | 4107 | 13874 | | | 41780 | |
| 687 | | | | | | | | | | |

TABLE V

| | Treated Animals: RsaI/HpaII Digest | | | | | | | | | |
|---|---|---|---|---|---|---|---|---|---|---|
| Base | Animal 6 | | Animal 7 | | Animal 8 | | Animal 9 | | Animal 10 | |
| Pairs | Replicate 1 | Replicate 2 | Replicate 1 | Replicate 2 | Replicate 1 | Replicate 2 | Replicate 1 | Replicate 2 | Replicate 1 | Replicate 2 |
| 101 | | | | | | | | | | |
| 107 | | | 3046 | 2054 | | 1766 | | | | 1679 |
| 114 | 2028 | 1800 | 1525 | 1830 | 2049 | 1435 | 1771 | 2052 | 2838 | |
| 146 | 1066 | | 11123 | 7048 | | | 3407 | 4545 | | 7298 |
| 147 | | | | | | | | | | |
| 148 | | | 4784 | 3370 | | | 2010 | | 1646 | 3761 |
| 163 | 3466 | 2742 | 40411 | 24825 | 3961 | | 14998 | 21395 | 5693 | 82588 |
| 173 | | | | 1711 | 2994 | 2060 | 1738 | 5156 | 3165 | |
| 185 | | | | | 3445 | 2083 | 1610 | 3678 | 3808 | |
| 191 | 3620 | 3005 | 43741 | 26582 | | | 15190 | 22359 | 3382 | 85172 |
| 201 | 8430 | 13506 | 12795 | 17273 | 34934 | 33377 | 31503 | 61970 | 60929 | 21185 |
| 208 | 3116 | 3439 | 1780 | 2919 | | 3479 | 3642 | 6518 | | |
| 210 | | 3684 | 7002 | 5968 | 6170 | 7050 | 5975 | 10912 | 7233 | 6517 |
| 213 | 3525 | 8858 | 10169 | 10177 | 26468 | 19616 | 18094 | 33607 | 42278 | 6483 |
| 221 | | | 11076 | 4778 | | | 1281 | 1231 | 2615 | 6006 |
| 238 | | | | | 3017 | 1880 | 1314 | 3527 | 704 | |
| 244 | | | | | 1390 | 2150 | 1893 | 5887 | 5606 | |
| 246 | | | | | 12180 | 1979 | 1671 | 3347 | 3008 | 1161 |
| 252 | | | 1127 | 802 | | | 506 | | 885 | 1753 |
| 271 | 2181 | 1632 | | | | | 3135 | 4447 | 2005 | 1757 |
| 273 | 3581 | 4117 | 3126 | 3844 | | | 8381 | 7459 | | 5042 |
| 275 | 1835 | 2952 | 2378 | 3024 | | | | | 13459 | 3727 |
| 277 | 5842 | 11052 | 5715 | 7380 | | | | | | 9228 |
| 278 | | | 8438 | 8973 | 150491 | 32953 | 32178 | 28731 | 25195 | 3646 |
| 279 | | | | | | | | | | |
| 284 | | | | 1323 | | 1158 | 1200 | 2250 | | 1221 |
| 287 | | 1809 | | 2216 | | 4588 | 4085 | 3223 | 5408 | |
| 289 | 5373 | 3519 | 23878 | 16177 | | 2212 | 10138 | 10587 | 6875 | 14367 |
| 295 | | | 2531 | 2262 | 2215 | | 1955 | 1643 | | 3412 |
| 315 | 234322 | 342536 | 314406 | 323703 | 420862 | 185404 | 194052 | 214350 | 216776 | |
| 325 | 193947 | 199399 | 251633 | 319940 | 263549 | 349372 | 363346 | 195630 | 313050 | 116687 |
| 342 | 4739 | 4650 | 3610 | 3333 | 4713 | 4718 | | | 4356 | |
| 358 | 188324 | 190413 | 200671 | 191370 | 202042 | 213722 | 211290 | 223538 | | |
| 362 | 265126 | 298444 | 325228 | 354333 | 359900 | 373231 | 345255 | 391354 | 482631 | 418748 |
| 381 | 7976 | 6216 | 11341 | 10464 | | 11129 | 16044 | 8369 | | |
| 404 | 14570 | 15269 | 12407 | 13708 | 2679 | 5080 | | 6804 | | |
| 406 | | | 7315 | 7778 | 2144 | | 5801 | 3581 | | |
| 408 | 5784 | 5628 | 13736 | 21138 | | 12682 | 7721 | 9145 | 1235 | |
| 445 | 177199 | 159259 | 184635 | 243329 | 79656 | 223593 | 220551 | 170720 | | |
| 479 | 20898 | 18501 | 26327 | 28546 | 5594 | 7280 | 21664 | 4846 | 4650 | |
| 498 | 62109 | 47369 | 72225 | 88416 | 36856 | 79182 | 83325 | 62694 | 13366 | |
| 513 | 6461 | 3387 | 3673 | 4060 | 7816 | 4046 | 2035 | 1284 | 2596 | |
| 555 | 57155 | 80317 | 123738 | 142565 | | 85696 | 112009 | | | |
| 566 | 51062 | 47493 | 59346 | 69739 | 14832 | 46651 | 37886 | 28254 | 13998 | |
| 580 | 10654 | 10550 | 22426 | 21640 | | 3310 | 10240 | | 7591 | |
| 600 | 11006 | 11774 | 26173 | 26208 | | | 18583 | 15027 | | |
| 602 | | 30724 | 69237 | 52118 | 5274 | 63239 | 63813 | 37426 | | |
| 687 | 4617 | 12022 | 10477 | 20530 | | 5829 | 11229 | 5268 | | |

To then compare changes between treated and control animals, a consensus control was created. For each PCR product size, an average peak area was determined across all 5 control samples. The standard deviation, standard error, and 95% confidence interval was also calculated and reported with each average. A consensus treated was also created using all 5 treated animals. The consensus treated was created using the same analysis steps indicated for creating a consensus control. The consensus treated peak areas were then calculated as a percent of the consensus control peak areas using the following equation: % HpaII consensus control=((HpaII consensus treated−HpaII consensus control)/HpaII consensus control)×100

The results were plotted using the Excel program as size of fragment in base pairs vs. percent consensus control. In this manner, all positives values indicate areas of hypermethylation while negative values represent areas of hypomethylation. Tables VI and VII show the calculated data for the consensus control and consensus treated.

TABLE VI

| | Control | | | | 95% |
|---|---|---|---|---|---|
| Base | Consensus | Number of | Standard | Standard | Confidence |
| Pairs | Average | observations | Deviation | Error | Interval |
| 101 | 1350 | 6 | 248 | 101 | 199 |
| 107 | 2224 | 8 | 528 | 187 | 366 |
| 114 | 2254 | 6 | 679 | 277 | 544 |
| 146 | | | | | |
| 147 | | | | | |
| 148 | | | | | |
| 163 | 7169 | 10 | 6915 | 2187 | 4286 |
| 173 | | | | | |
| 185 | | | | | |
| 191 | 7550 | 10 | 7619 | 2409 | 4722 |
| 201 | 18731 | 10 | 10081 | 3188 | 6248 |
| 208 | 3858 | 6 | 1280 | 523 | 1024 |
| 210 | 3684 | 6 | 854 | 349 | 684 |
| 213 | 6665 | 8 | 2893 | 1023 | 2005 |
| 221 | 2850 | 6 | 2119 | 865 | 1696 |
| 238 | | | | | |
| 244 | 3013 | 7 | 962 | 364 | 713 |
| 246 | | | | | |
| 252 | | | | | |
| 271 | 2945 | 6 | 627 | 256 | 502 |
| 273 | 6548 | 7 | 2291 | 866 | 1697 |
| 275 | 3531 | 6 | 1129 | 461 | 903 |
| 277 | 14864 | 9 | 10433 | 3478 | 6816 |
| 278 | 8391 | 7 | 8680 | 3281 | 6430 |
| 279 | 14981 | 6 | 5835 | 2382 | 4669 |
| 284 | 2199 | 6 | 982 | 401 | 786 |
| 287 | | | | | |
| 289 | 2723 | 9 | 1579 | 526 | 1032 |
| 295 | | | | | |
| 315 | 285305 | 7 | 98617 | 37274 | 73055 |
| 325 | 142968 | 9 | 98999 | 33000 | 64678 |
| 342 | | | | | |
| 358 | 186162 | 8 | 78320 | 27690 | 54272 |
| 362 | 349416 | 8 | 83603 | 29558 | 57933 |
| 381 | 6371 | 7 | 8178 | 3091 | 6058 |
| 404 | | | | | |
| 406 | | | | | |
| 408 | 6345 | 7 | 2398 | 906 | 1777 |
| 445 | 94565 | 9 | 48185 | 16062 | 31480 |
| 479 | 21203 | 5 | 15346 | 6863 | 13451 |
| 498 | 39405 | 9 | 19615 | 6538 | 12815 |
| 513 | 4118 | 6 | 1444 | 590 | 1155 |
| 555 | 51277 | 5 | 59276 | 26509 | 51957 |
| 566 | 18635 | 9 | 17702 | 5901 | 11565 |
| 580 | 12350 | 5 | 10970 | 4906 | 9616 |
| 600 | 7353 | 8 | 5923 | 2094 | 4104 |
| 602 | 13924 | 5 | 18602 | 8319 | 16305 |
| 687 | | | | | |

TABLE VII

| | Treated | | | | 95% | | Opposite of |
|---|---|---|---|---|---|---|---|
| Base | Consensus | Number of | Standard | Standard | Confidence | % Consensus | % Consensus |
| Pairs | Average | observations | Deviation | Error | Interval | Control | Control |
| 101 | | | | | | −100.0 | 100.0 |
| 107 | 2136 | 6 | 654 | 267 | 523 | −3.9 | 3.9 |
| 114 | 1925 | 9 | 483 | 161 | 315 | −14.6 | 14.6 |
| 146 | 5748 | 7 | 3811 | 1440 | 2823 | | |
| 147 | | | | | | | |
| 148 | 3114 | 5 | 1275 | 570 | 1117 | | |
| 163 | 22231 | 9 | 16481 | 5494 | 10767 | 210.1 | −210.1 |
| 173 | 2804 | 7 | 708 | 268 | 524 | | |
| 185 | 2925 | 5 | 851 | 380 | 746 | | |
| 191 | 25381 | 8 | 17706 | 6260 | 12269 | 236.2 | −236.2 |
| 201 | 29590 | 10 | 16765 | 5302 | 10391 | 58.0 | −58.0 |
| 208 | 3556 | 8 | 1142 | 404 | 792 | −7.8 | 7.8 |
| 210 | 6723 | 10 | 1276 | 404 | 791 | 82.5 | −82.5 |
| 213 | 17928 | 10 | 11087 | 3506 | 6872 | 169.0 | −169.0 |
| 221 | 4498 | 6 | 3978 | 1624 | 3183 | 57.8 | −57.8 |
| 238 | 2088 | 5 | 992 | 444 | 869 | | |
| 244 | 3385 | 5 | 1946 | 870 | 1706 | 12.3 | −12.3 |
| 246 | 3891 | 6 | 4130 | 1686 | 3304 | | |
| 252 | 1015 | 5 | 329 | 147 | 288 | | |
| 271 | 2526 | 6 | 872 | 356 | 698 | −14.2 | 14.2 |
| 273 | 5079 | 8 | 2304 | 815 | 1597 | −22.4 | 22.4 |
| 275 | 4563 | 6 | 4983 | 2034 | 3987 | 29.2 | −29.2 |
| 277 | 7843 | 6 | 1630 | 666 | 1305 | −47.2 | 47.2 |
| 278 | 36326 | 8 | 50240 | 17763 | 34814 | 332.9 | −332.9 |
| 279 | | | | | | −100.0 | 100.0 |
| 284 | 1430 | 8 | 231 | 82 | 160 | −35.0 | 35.0 |
| 287 | 3555 | 9 | 1433 | 478 | 936 | | |
| 289 | 10347 | 10 | 7424 | 2348 | 4601 | 280.0 | −280.0 |
| 295 | 2336 | 7 | 526 | 199 | 389 | | |
| 315 | 271823 | 9 | 74234 | 24745 | 48499 | −4.7 | 4.7 |
| 325 | 256655 | 10 | 50999 | 16127 | 31609 | 79.5 | −79.5 |
| 342 | 4303 | 7 | 526 | 199 | 390 | | |
| 358 | 202671 | 8 | 9480 | 3352 | 6569 | 8.9 | −8.9 |
| 362 | 361425 | 10 | 61861 | 19562 | 38341 | 3.4 | −3.4 |
| 381 | 10220 | 8 | 2865 | 1013 | 1985 | 60.4 | −60.4 |
| 404 | 10074 | 8 | 5179 | 1831 | 3589 | | |
| 406 | 5324 | 5 | 2403 | 1075 | 2106 | | |
| 408 | 9634 | 9 | 5840 | 1947 | 3815 | 51.8 | −51.8 |
| 445 | 182368 | 8 | 47923 | 16943 | 33208 | 92.8 | −92.8 |
| 479 | 15367 | 9 | 9523 | 3174 | 6221 | −27.5 | 27.5 |
| 498 | 60616 | 9 | 27062 | 9021 | 17680 | 53.8 | −53.8 |
| 513 | 3929 | 9 | 2014 | 671 | 1316 | −4.6 | 4.6 |
| 555 | 100247 | 7 | 24186 | 9141 | 17917 | 95.5 | −95.5 |
| 566 | 41029 | 9 | 19325 | 6442 | 12625 | 120.2 | −120.2 |
| 580 | 12344 | 8 | 6881 | 2433 | 4768 | 0.0 | 0.0 |
| 600 | 18129 | 6 | 6376 | 2603 | 5102 | 146.5 | −146.5 |
| 602 | 45976 | 8 | 22789 | 8057 | 15792 | 230.2 | −230.2 |
| 687 | 9996 | 8 | 3325 | 1176 | 2304 | | |

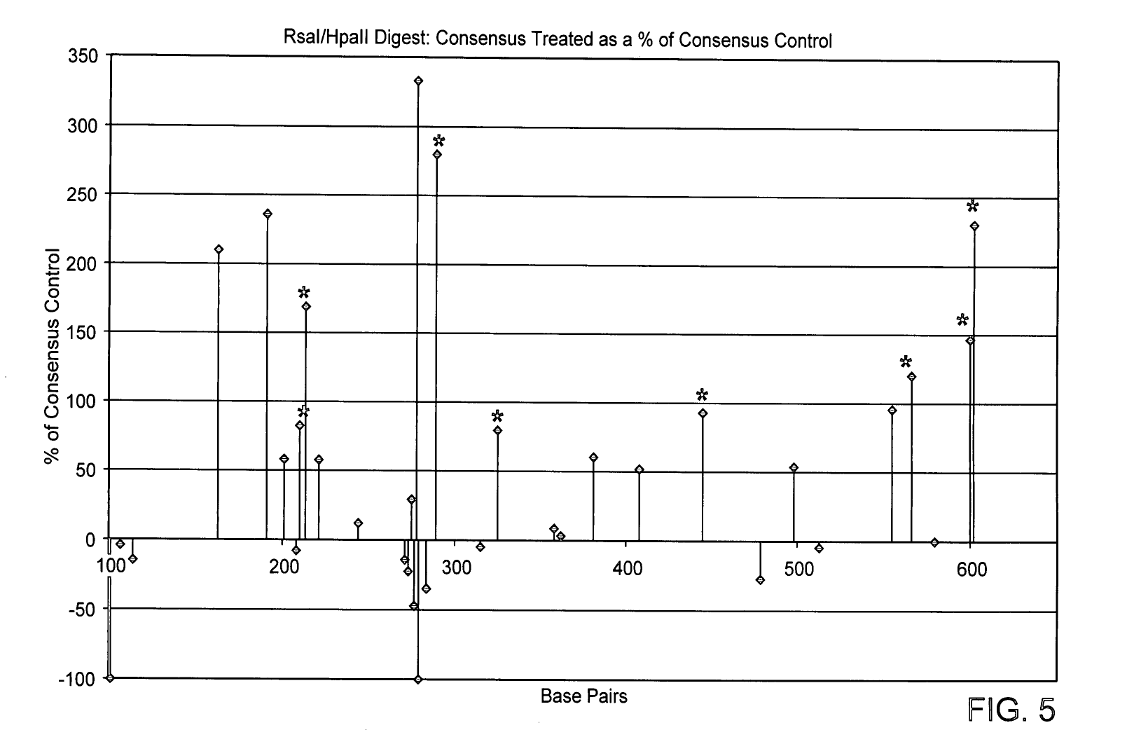

FIG. 5 shows RsaI/HpaII digest, following arbitrarily primed PCR (AP-PCR) as capillary electrophoresis (CE) data output plotted as size in base pairs vs. percent consensus control (where the consensus treated is shown as a percent of the consensus control). All positive values indicated less cutting by HpaII. This decreased cutting by HpaII indicated that there was hypermethylation at the internal cytosine in the 5′CCGG 3′ recognition sequence. All negative values thus indicate more cutting by HpaII. This increased cutting indicates there was hypomethylation at the internal cytosine in the 5′CCGG3′ recognition sequence. The red asterisks denote a statistically significant difference between the control mean and treated mean for that size PCR product found by conducting a t-test where α=0.05. With this method, 8 sites of hypermethylation (positive values with red asterisks) in the treated animals in response to the Phenobarbital treatment were identified. Additionally, two sites that were methylated in the control were found to be completely hypomethylated (−100%) in the treated. Therefore, using the methods of the invention, 10 sites in murine liver DNA were identified where phenobarbital treatment has affected methylation status.

Further Studies on GC-Rich Region Methylation Following Phenobarbital Treatment

In a further investigation of the effects of the non-genotoxic rodent carcinogen phenobarbitol (PB), 6-7 wk old male B6C3F1 mice (6-7 wks) were administered 0.05% (w/v) phenobarbital in their drinking water for 2 weeks. The mice were sacrificed at 2 weeks and livers were snap frozen in liquid nitrogen. DNA was isolated from the liver tissue using the TRIzol Reagent (Invitrogen). In order to assess changes in the methylation of GC-rich regions in the livers of treated and control mice, genomic DNA was analyzed by arbitrarily primed-PCR and capillary electrophoresis.

Each liver DNA sample was digested with RsaI and HpaII, or RsaI and MspI concurrently. RsaI recognizes the sequence 5′GTAC 3′ and is used to cut the DNA into manageable size fragments. Both HpaII and MspI recognize the recognition sequence of 5′CCGG 3′. In general, MspI will not cut the site if the external (5′) cytosine is methylated, and HpaII will not cut the site if the internal (3′) cytosine is methylated. PCR was performed on the digested samples using an arbitrary primer labeled on the 5′ end with HEX™ (hexachlorofluorescein). The PCR products were the desalted using the Qiagen PCR purification kit. Ten nanograms of each purified PCR product were added to a mixture of formamide and a carboxy-X-rodamine (ROX™)-labeled 1000 bp size marker. 2 ul of this mixture was injected for caplillary electrophoresis using a 10 sec injection time.

Peak area averages were calculated for the control group at each PCR fragment size for a particular digest (RsaI/HpaII or RsaI/MspI). To compare changes between animals administered PB (treated) and control animals, each treated sample was calculated as a percent of the averaged control using the formular: % MspI Control=(MspI treated−MspI averaged control)/MspI averaged control)×100, and % HpaII Control=(HpaII treated−HpaII averaged control)/HpaII averaged control)×100. The results were plotted using Excel as size of fragment in base pairs versus percent averaged control (not shown). Statistical significance was determined using Student's t-test.

The results of the RsaI/HpaII analysis showed that phenobarbital treatment yielded 5 sites of hypomethylation, 10 sites of hypermethylation, and 11 sites of new methylation. The results of RsaI/MspI analysis showed that phenobarbital treatment yielded 8 sites of hypomethylation, 8 sites of hypermethylation, and 11 sites of new methylation.

Table VIII is a summary of the numbers of altered sites detected. This table provides a comparison of GC-rich methylation sites of change in control and Phenobarbital-treated B6C3F1 mice

TABLE VIII

| | | | | Sites of | |
|---|---|---|---|---|---|
| | | | | “New” | |
| Treat- | Di- | Sites of | Sites of | Methyl- | |
| ment | gest | Hypomethylation | Hypermethylation | ation | TOTAL |
| 0.05% | HpaII | 5 | 10 | 11 | 26 |
| | MspI | 8 | 8 | 11 | 27 |
| Total | | 13 | 18 | 22 | 53 |

The results show that changes in the methylation status of GC-rich genomic regions, including hypomethylations, hypermethylations, and new methylations, occur during, and may be involved in the development and progression of liver tumorigenesis.

6.4. Methylation Status Changes During in Early Stage Skin Tumorogenesis

Gene-specific changes in methylation have been detected during skin tumor promotion (see Watson, et al. (2004) Mol. Car. 41: 54-66). In order to analyze changes in genome-wide methylation status occurring during the promotion stage of skin tumorogenesis, female SENCAR mice (5-7 wks old) were selectively bred to be sensitive to skin carcinogenesis. These mice form skin tumors when subjected to an initiation/promotion model.

Mice were initiated with a single application of 75 μg DMBA (7,12-dimethylbenz [α]anthracene), and then promoted with thrice weekly applications of 27 mg of cigarette smoke condensate (CSC) for 8 weeks. Control mice were promoted with the vehicle, acetone. DNA was isolated from the skin tissue at the site of application using the TRI□ Reagent (Sigma).

GC-rich methylation was then assessed with arbitrarily primed-PCR and capillary electrophoresis. Each skin DNA sample was digested with RsaI and HpaII OR RsaI and MspI concurrently. RsaI recognizes the sequence 5′GTAC 3′ and is used to cut the DNA into manageable size fragments. Both HpaII and MspI recognize the recognition sequence of 5′CCGG 3′. In general, MspI will not cut the site if the external (5′) cytosine is methylated and HpaII will not cut the site if the internal (3′) cytosine is methylated. PCR was performed on the digested samples using an arbitrary primer labeled on the 5′ end with hexachlorofluoresceins (HEX™) and PCR products were desalted using the Qiagen PCR purification kit. Ten nanograms of each purified PCR product were then added to a mixture of formamide and a carboxy-X-rodamine-(ROX™)-labeled 1000 bp size marker. 2 ul of this mixture was injected for electrophoresis using a 10 sec injection time.

In order to analyze the results, rodamine fluorescence was measured throughout the electrophoretic separation and peak area averages were calculated for the control group at each PCR fragment size for a particular digest (RsaI/HpaII or RsaI/MspI). To compare changes between samples promoted with CSC (treated) and those promoted with acetone (control), each treated sample was calculated as a percent of the averaged control using the formulas: % MspI Acetone Control=(MspI treated−MspI averaged control)/MspI averaged control)×100; and % HpaII Acetone Control=(HpaII treated−HpaII averaged control)/HpaII-averaged control)×100.

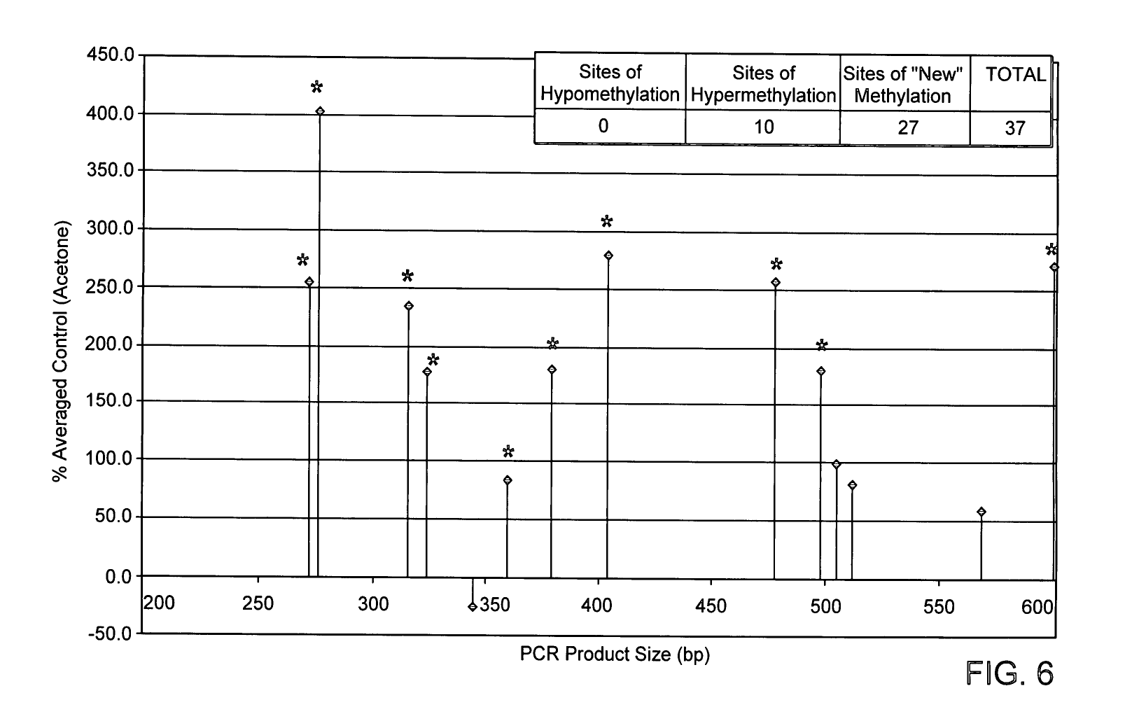

The results were then plotted using Excel as size of fragment in base pairs versus percent averaged control. The results from Rsa/HpaII and RsaI/MspI digests for promotion with 27 mg CSC are shown in FIGS. 6-9, which are described in detail below.

FIG. 6 is a graph showing the effects of high dose (27 mg CSC) promotion on the methylation of GC rich regions. RsaI/MspI digest, arbitrarily primed PCR and capillary electrophoresis was performed on DNA isolated from SENCAR control (Acetone) or treated (27 mg CSC) mice. Promotion with 27 mg CSC for 8 wks yielded 10 sites of hypermethylation and 27 sites of new methylation. Positive values indicate sites of hypermethylation while negative values indicate sites of hypomethylation. The asterisks denote a significant difference between the control mean and treated mean for that size PCR product where p<0.05 in the Student's t-test.

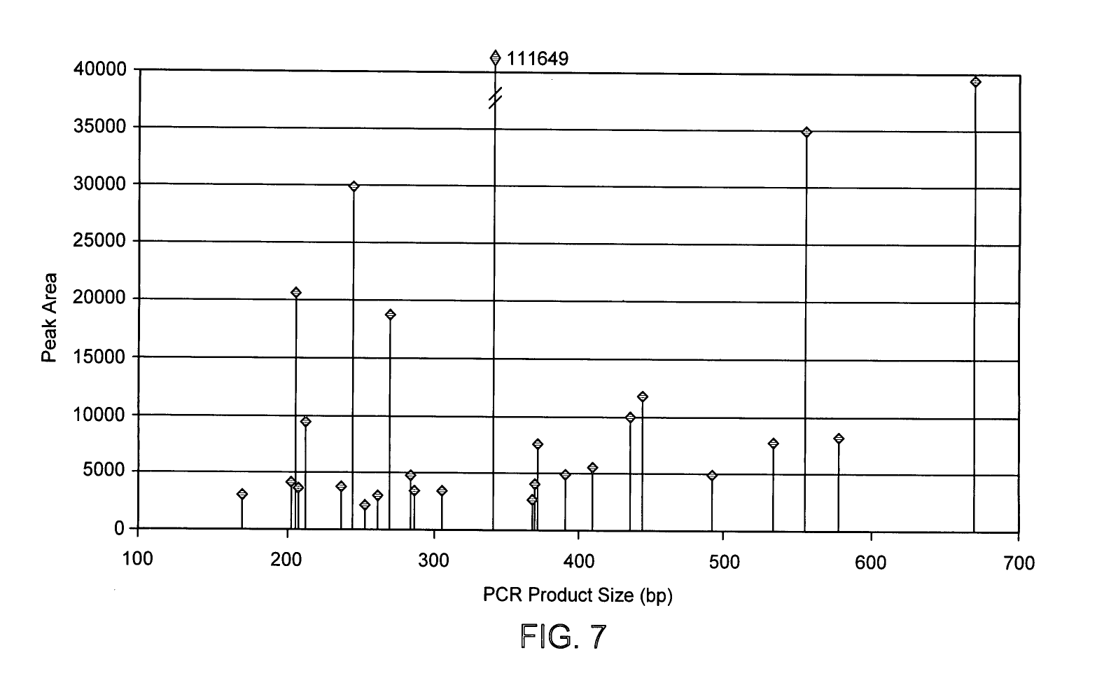

FIG. 7 is a graph showing sites of new methylation following high dose promotion (27 mg CSC). RsaI/MspI digest, arbitrarily primed PCR and capillary electrophoresis was performed on DNA isolated from SENCAR control (Acetone) or treated (27 mg CSC) mice. Promotion with 27 mg CSC for 8 wks yielded 27 sites of new methylation. Data are expressed in terms of the peak area for each PCR product size. One Peak area exceeded the chart scale and was included above the chart for reference—the actual peak area value is listed next to that data point.

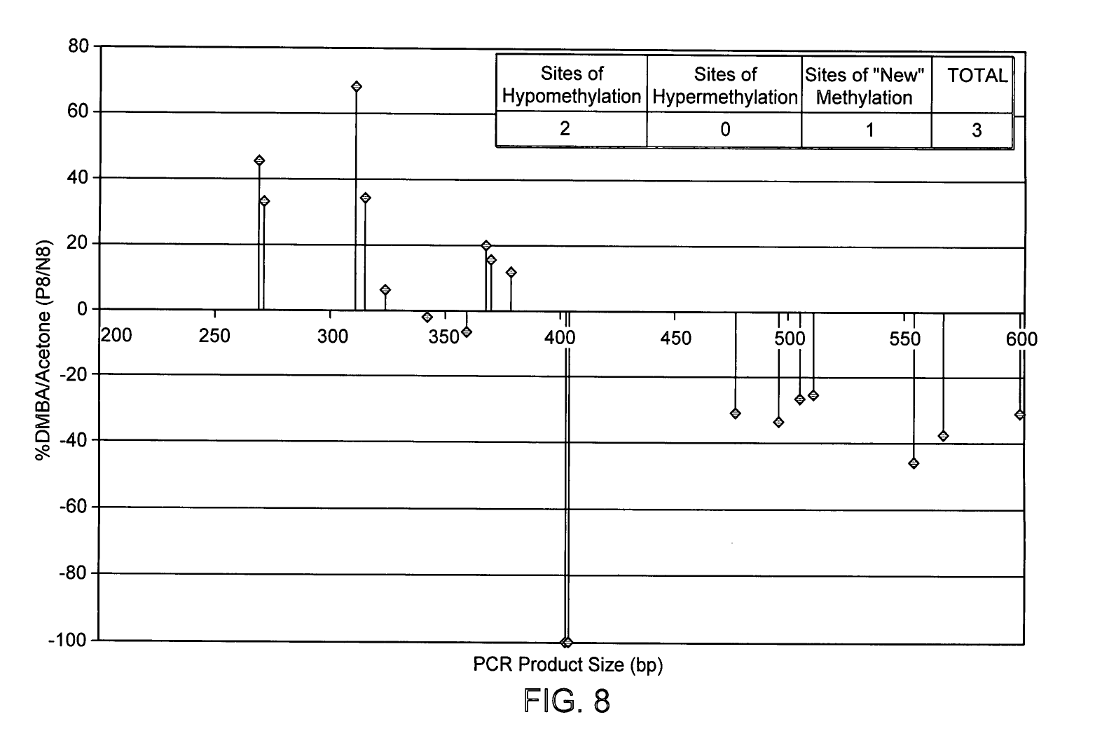

FIG. 8 is a graph showing the effects of high dose (27 mg CSC) promotion on GC rich region methylation. RsaI/HpaII digest, arbitrarily primed PCR and capillary electrophoresis was performed on DNA isolated from SENCAR control (Acetone) or treated (27 mg CSC) mice. Promotion with 27 mg CSC for 8 wks yielded 2 sites of hypomethylation and 1 site of new methylation. Positive values indicate sites of hypermethylation while negative values indicate sites of hypomethylation. Diamonds denote a significant difference between the control mean and treated mean for that size PCR product where p<0.05. Statistical significance was determined using Student's t-test.

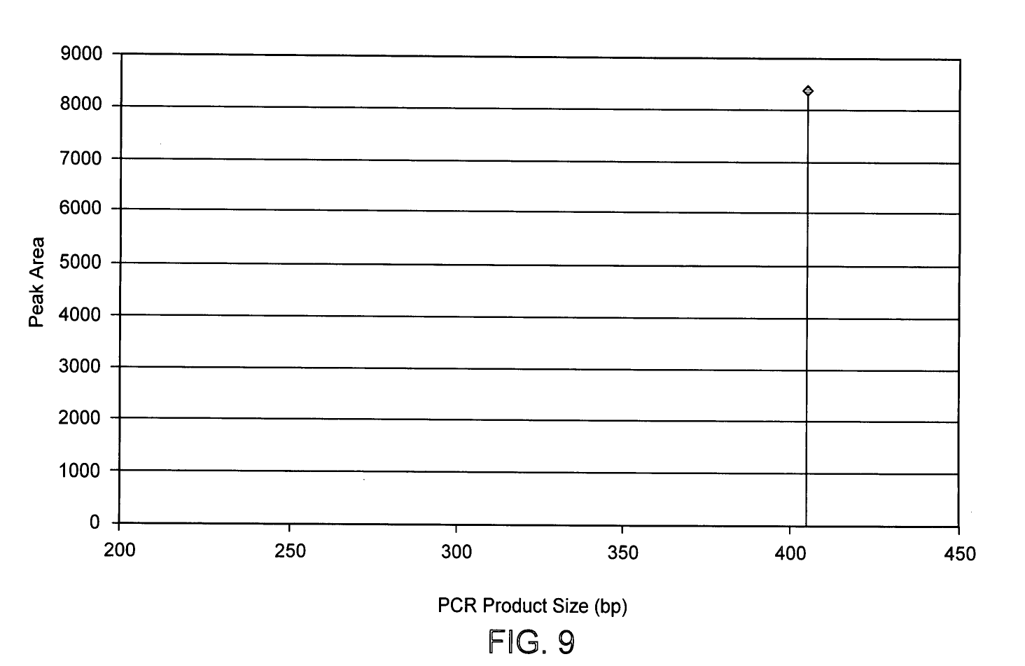

FIG. 9 is a graph showing the site of new methylation following high dose promotion (27 mg CSC). RsaI/HpaII digest, Arbitrarily primed PCR and capillary electrophoresis was performed on DNA isolated from SENCAR control (Acetone) or treated (27 mg CSC) mice. Promotion with 27 mg CSC for 8 wks yielded 1 site of new methylation. Data are expressed in terms of the peak area for each PCR product size.

Table IX is a summary of the numbers of altered sites detected. This table provides a comparison of DMBA initiated, 27 mg CSC eight week skin tumor promotion to an eight week acetone mock promotion.

TABLE IX

| | | | | Sites of | |
|---|---|---|---|---|---|
| | | Sites | Sites of | “New” | |
| Treatment | Digest | of Hypomethylation | Hypermethylation | Methylation | TOTAL |
| 27 mg CSC | HpaII | 2 | 0 | 1 | 3 |
| | MspI | 0 | 10 | 27 | 37 |
| Total | | 2 | 10 | 28 | 40 |

The results show that changes in the methylation status of GC-rich genomic regions, including hypomethylations, hypermethylations, and new methylations, occur in the process of the promotion of skin tumorogenesis.

6.5. Methylation Status Changes in Hypertensive Aorta

Changes in DNA methylation have been associated with atherosclerosis, a degenerative condition affecting arteries in which there is hyperplasia of the outer coat and fatty degeneration of the middle coat of the arteries due to the formation of plaques in the inner lining of the artery. In particular, DNA hypomethylation has been shown to be associated with atherogenic vascular disease (Castro et al. (2003) Clin. Chem. 49: 1292-6).

Because of the cellular proliferation and monoclonality of at least some of the lesion cells, atherosclerotic lesions have been compared with benign vascular tumors (see Penn et al. (1986) Proc. Natl. Acad. Sci. 83: 7951-55). In order to investigate the possibility that DNA methylation changes are associated with diseases that are clearly distinct from cancers, the effect of hypertension on the methylation status of GC-rich regions of genomic DNA was investigated using aortas isolated from hypertensive rats.

Hypertensive rats were created by subjecting male Sprague-Dawley rats, weighing 250-300 g to a uninephrectomy and implanting them with a deoxycorticosterone acetate (DOCA) pellet (200 mg/kg). DOCA-treated rats received water supplemented with 1.0% NaCl and 0.2% KCl. Aortas were removed 28 days after implantation of the DOCA pellet. At 28 days, systolic blood pressure is 112 mmHg and 180 mmHg in control and DOCA-treated rats, respectively.

DNA from the control and hypertensive rat aortas was then extracted and analyzed. Each genomic DNA sample was digested with RsaI and HpaII OR RsaI and MspI concurrently. RsaI recognizes the sequence 5′GTAC 3′ and is used to cut the DNA into manageable size fragments, while both HpaII and MspI recognize the recognition sequence of 5′CCGG 3′. In general MspI does not cut the site if the external (5′) cytosine is methylated, and HpaII will not cut the site if the internal (3′) cytosine is methylated. PCR was then performed on the digested samples using an arbitrary primer labeled on the 5′ end with HEX™ (hexachlorofluorescein). PCR products were desalted using the Qiagen PCR purification kit, and 10 ng of each purified PCR product was added to a mixture of formamide and a carboxy-X-rodamine (ROX™)-labeled 1000 bp size marker. 2 ul of this mixture was injected for electrophoresis using a 10 sec injection time.

In order to analyze the results, rodamine fluorescence was measured throughout the electrophoretic separation and peak area averages were calculated for the control group at each PCR fragment size for a particular digest (RsaI/HpaII or RsaI/MspI). To compare changes between hypertensive (treated) aortas and those normal (control) aortas, each treated sample was calculated as a percent of the averaged control using the formulas % MspI Control=(MspI treated−MspI averaged control)/MspI averaged control)×100, and % HpaII Control=(HpaII treated−HpaII averaged control)/HpaII averaged control)×100

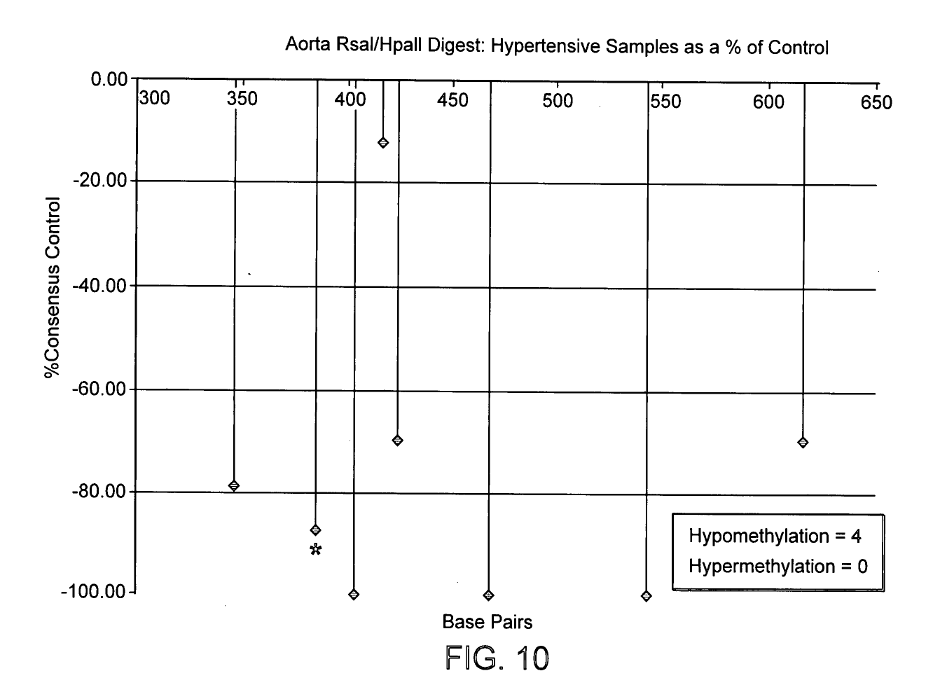

Results are plotted using Excel as size of fragment in base pairs versus percent averaged control. The results from Rsa/HpaII and RsaI/MspI digests for hypertensive aortas, as compared to control aortas, are shown in FIGS. 10-13, which are described in detail below.

FIG. 10 is a graph showing the effect of hypertension on the methylation status of GC-rich regions of DNA. RsaI/HpaII digest, arbitrarily primed PCR and capillary electrophoresis was performed on DNA isolated from the aortas of control and hypertensive rats. The data are expressed in terms of the hypertensive mean (consensus hypertensive) for each PCR product size as a percent of the control mean (consensus control) for each PCR product size. Positive values indicate sites of hypermethylation while negative values indicate sites of hypomethylation. The results show four significant sites of hypomethylation in hypertensive aortas as compared to control aortas. Only those values that are significantly different from control are considered to be “changes,” Student's t-test, p<0.05.

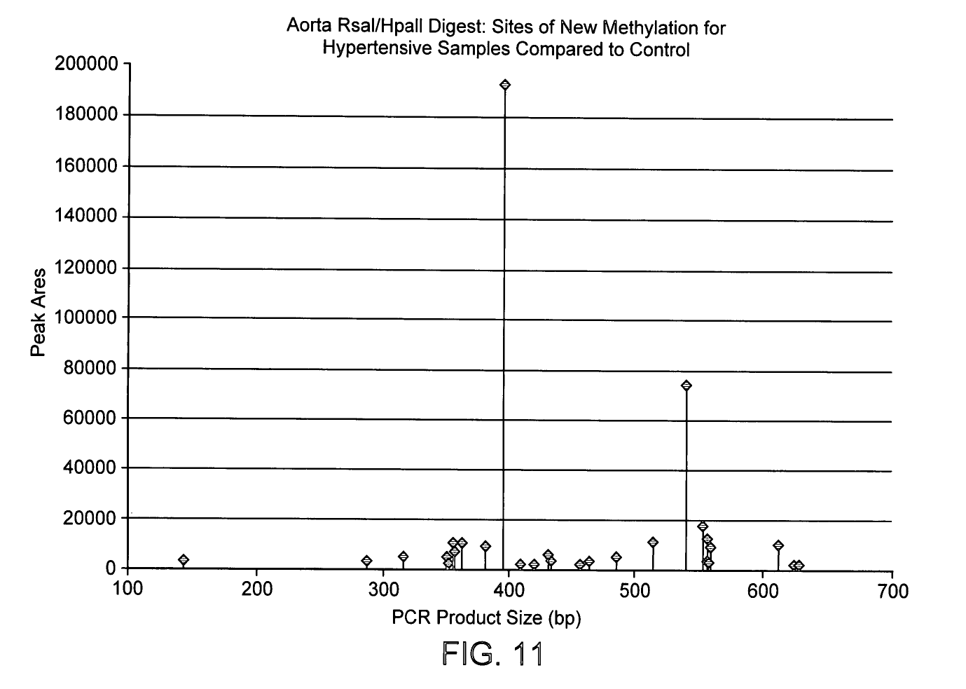

FIG. 11 shows the effect of hypertension on the methylation status of GC-rich regions of DNA. Sites of new methylation were investigated using an RsaI/HpaII digest and subsequent AP-PCR, followed by separation of the products by capillary electrophoresis, on DNA isolated from the aortas of control and hypertensive rats. The data presented indicate sites of new methylation, i.e., sites that were methylated in the treated animals but not in the controls.

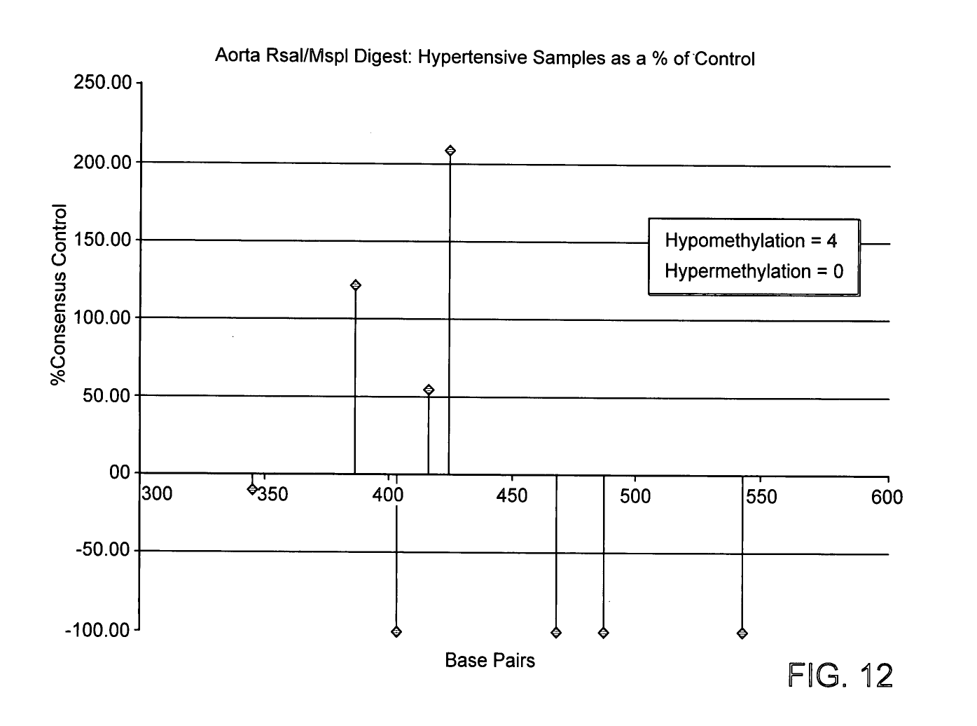

FIG. 12 shows the effect of hypertension on the methylation status of GC-rich regions of DNA. Sites of hypomethylation and hypermethylation associated with hypertension were investigated using a RsaI/MspI digest. An RsaI/MspI digest and subsequent AP-PCR, followed by separation of the products by capillary electrophoresis, was performed on DNA isolated from the aortas of control and hypertensive rats. The data is expressed in terms of the hypertensive mean (consensus hypertensive) for each PCR product size as a percent of the control mean (consensus control) for each PCR product size. Positive values indicate sites of hypermethylation while negative values indicate sites of hypomethylation. The results show four significant sites of hypomethylation in hypertensive aortas as compared to control aortas. Only those values that are significantly different from control are considered to be “changes,” Student's t-test, p<0.05.

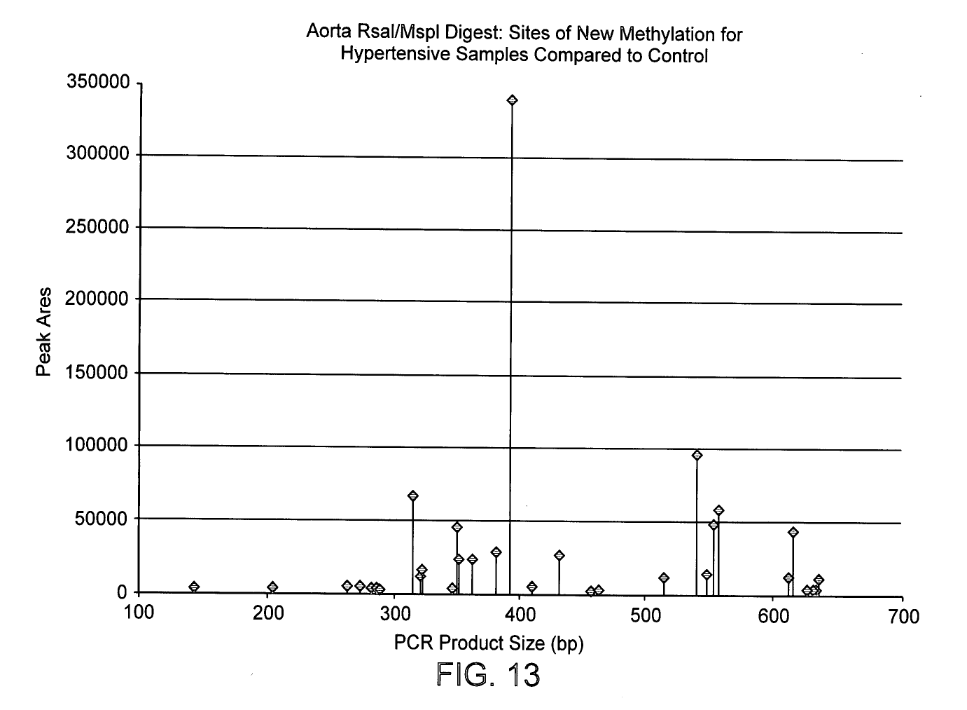

FIG. 13 shows the effect of hypertension on the methylation status of GC-rich regions of DNA. Sites of new methylation were investigated using an RsaI/MspII digest. An RsaI/MspI digest and subsequent AP-PCR, followed by separation of the products by capillary electrophoresis, was performed on DNA isolated from the aortas of control and hypertensive rats. The data presented indicate sites of new methylation, i.e., sites that were methylated in the treated animals but not in the controls.

Table X is a summary of the changes in GC rich methylation sites in hypertensive versus control rat aortas.

TABLE X

| | | | | Sites of | |
|---|---|---|---|---|---|
| | | Site | Sites of | “New” | |
| Group | Digest | of Hypomethylation | Hypermethylation | Methylation | Total |
| Hypertensive | RsaI/HpaII | 4 | 0 | 30 | 34 |
| Rat Aora | | | | | |
| Hypertensive | RsaI/MspI | 4 | 0 | 33 | 37 |
| Rat Aora | | | | | |
| Totals | | 8 | 0 | 63 | 71 |

The results show that the methylation status of DNA in the aorta is altered in hypertensive rats. The most frequent change observed is an increase in “new” methylations, i.e., sites of methylation observed in the hypertensive animals and not in the controls. Accordingly, changes in methylation status appear to be involved in hypertension, a vascular disease with complex origins that does not appear to be associated with cancer-like cell hyperplasia.

### EQUIVALENTS

As will be apparent to those skilled in the art to which the invention pertains, the present invention may be embodied in forms other than those specifically disclosed above without departing from the spirit or essential characteristics of the invention. The particular embodiments of the invention described above, are, therefore, to be considered as illustrative and not restrictive. The scope of the invention is as set forth in the appended claims rather than being limited to the examples contained in the foregoing description.

1. A method for determining the methylation status of a target double-stranded nucleic acid molecule, comprising:
(a) contacting the target double-stranded nucleic acid molecule with a methylation-sensitive restriction endonuclease under conditions wherein the target double-stranded nucleic acid molecule is cleaved at a site recognized by the methylation-sensitive restriction endonuclease if the site is not methylated;
(b) PCR amplifying the product of step (a) with a detectably labeled primer that hybridizes to a predetermined region of a strand of the double-stranded nucleic acid molecule; and
(c) detecting the presence of a product of step (b) using capillary electrophoresis,
wherein the presence a product in step (c) indicates that the target double-stranded nucleic acid molecule is methylated at the site recognized by the methylation-sensitive restriction endonuclease.

2. The method of claim 1, wherein step (b) further comprises PCR amplifying with a second primer that hybridizes to a second predetermined region of a second strand of the double-stranded nucleic acid molecule.

3. The method of claim 1, wherein the target double-stranded nucleic acid molecule is isolated from a cell.

4. The method of claim 3, wherein the cell is a mammalian cell.

5. The method of claim 3, wherein the target double-stranded nucleic acid molecule is genomic DNA.

6. The method of claim 3, wherein the target double-stranded nucleic acid molecule is heterologous to the cell.

7. The method of claim 1, wherein the predetermined region is a GC-rich region.

8. The method of claim 1, wherein the predetermined region is a region at the 3′ end of one of the strands of the double-stranded nucleic acid molecule.

9. The method of claim 1, wherein the detectably labeled primer is labeled with a fluorophore.

10. A method for determining the methylation status of a double-stranded nucleic acid molecule, comprising:
(a) contacting the double-stranded nucleic acid molecule with a methylation-sensitive restriction endonuclease under conditions wherein the double-stranded nucleic acid molecule is cleaved at a site recognized by the methylation-sensitive restriction endonuclease if the site is not methylated;
(b) PCR amplifying the product of step (a) with a detectably labeled primer that hybridizes to a predetermined region of a strand of the double-stranded nucleic acid molecule;
(c) detecting the presence of a product of step (b) using capillary electrophoresis;
(d) contacting the double-stranded nucleic acid molecule with a methylation-insensitive restriction endonuclease under conditions wherein the double-stranded nucleic acid molecules is cleaved at the same site recognized by the methylation-sensitive restriction endonuclease;
(e) PCR amplifying the product of step (d) with the detectably labeled primer;
(f) detecting the presence of a product of step (e) using capillary electrophoresis; and
(d) comparing the result of step (c) with a result of step (f),
wherein a difference in the results obtained from steps (c) and (f) indicates that the double-stranded nucleic acid molecule is methylated at the site recognized by the methylation-sensitive restriction endonuclease.

11. The method of claim 9, wherein the difference is an increase in the number of products in step (c) as compared to the number of products in step (f).

12. The method of claim 10, wherein the predetermined region is a GC-rich region.

13. The method of claim 10, wherein the nucleic acid molecule is genomic DNA isolated from a cell.

14. The method of claim 11, wherein the cell has been contacted with a compound.

15. The method of claim 11, wherein the cell is a mammalian cell.

16. The method of claim 10, wherein the detectably labeled primer is labeled with a fluorophore.

17. A method for determining if a compound affects the methylation status of a cell, comprising:
(a) contacting a cell with a compound
(b) isolating a double-stranded nucleic acid molecule from the contacted cell;
(c) contacting the double-stranded nucleic acid molecule with a methylation-sensitive restriction endonuclease under conditions wherein the double-stranded nucleic acid molecule is cleaved at a site recognized by the methylation-sensitive restriction endonuclease if the site is not methylated;
(d) PCR amplifying the product of step (c) with a detectably labeled primer that hybridizes to a predetermined region of a strand of the double-stranded nucleic acid molecule;
(e) detecting the presence of a product of step (d) using capillary electrophoresis;
(f) performing steps (c)-(e) with a double-stranded nucleic acid molecule isolated from a cell not contacted with the compound; and
(g) comparing the result obtained from step (e) with the result obtained from step (f),
wherein a difference in the results obtained from steps (e) and (f) indicates that the compound affects the methylation status of a cell.

18. The method of claim 17, wherein the predetermined region is a GC-rich region.

19. The method of claim 17, wherein the difference is an increase in the number of products in step (e) as compared to the number of products in step (f).

20. The method of claim 17, wherein the compound abrogates the growth of the cell.

21. The method of claim 20, wherein the compound is toxic to the cell.

22. The method of claim 17, wherein the difference is a decrease in the number of products in the result of step (e) as compared to the number of products in the result of step (f).

23. The method of claim 17, wherein the compound enhances the proliferation of the cell.

24. The method of claim 23, wherein the compound is a carcinogen.

25. The method of claim 17, wherein the detectably labeled primer is labeled with a fluorophore.

26. A method for determining the level of expression of a target nucleic acid molecule by a cell, comprising:
(a) contacting the target double-stranded nucleic acid molecule isolated from the cell with a methylation-sensitive restriction endonuclease under conditions wherein the target double-stranded nucleic acid molecule is cleaved at a site recognized by the methylation-sensitive restriction endonuclease if the site is not methylated;
(b) PCR amplifying the product of step (a) with a detectably labeled primer that that hybridizes to a predetermined region of a strand of the double-stranded nucleic acid molecule; and
(c) detecting the presence of a product of step (b) using capillary electrophoresis,
wherein the number of products in step (c) is inversely related to the level of expression of the target double-stranded nucleic acid molecule by the cell.

27. The method of claim 26, wherein step (b) further comprises PCR amplifying with a second primer that hybridizes to a second predetermined region of a second strand of the double-stranded nucleic acid molecule.

28. The method of claim 26, wherein the target double-stranded nucleic acid molecule is isolated from a cell.

29. The method of claim 28, wherein the cell is a mammalian cell.

30. The method of claim 29, wherein the target double-stranded nucleic acid molecule is heterologous to the cell.

31. The method of claim 26, wherein the predetermined region is a region at the 3′ end of one of the strands of the double-stranded nucleic acid molecule.

32. The method of claim 26, wherein the detectably labeled primer is labeled with a fluorophore.


SEQUENCE LISTING (5 sequences)

SEQ ID NO: 1 - DNA, 20 bp, Artificial Sequence
  Description of Artificial Sequence Synthetic primer
aaccctcaccctaaccccgg

SEQ ID NO: 2 - DNA, 19 bp, Artificial Sequence
  Description of Artificial Sequence Synthetic primer
taactccatccaacccggg

SEQ ID NO: 3 - DNA, 16 bp, Artificial Sequence
  Description of Artificial Sequence Synthetic primer
aacccctaatcccggg

SEQ ID NO: 4 - DNA, 14 bp, Artificial Sequence
  Description of Artificial Sequence Synthetic primer
acctcccaatgcgc

SEQ ID NO: 5 - DNA, 17 bp, Artificial Sequence
  Description of Artificial Sequence Synthetic primer
cattctaccccatgcgc